(19) United States
(12) Patent Application Publication
(10) Pub. No.: US 2026/0116856 A1
(43) Pub. Date: Apr. 30, 2026

(54) METHOD FOR PREPARING META-(CHIRAL ALLYL)-SUBSTITUTED PYRIDINE COMPOUND

(71) Applicant: NANKAI UNIVERSITY, Tianjin (CN)

(72) Inventors: Xiaochen WANG, Tianjin (CN); Zhujun SHI, Tianjin (CN); Zhong LIU, Tianjin (CN); Junjie TIAN, Tianjin (CN); Ruirui LI, Tianjin (CN)

(73) Assignee: NANKAI UNIVERSITY, Tianjin (CN)

(21) Appl. No.: 19/476,673

(22) PCT Filed: Jun. 15, 2023
(86) PCT No.: PCT/CN2023/100491

§ 371 Date: Oct. 17, 2025

(30) Foreign Application Priority Data
Apr. 18, 2023 (CN) 202310414762.5

(51) Int. Cl. C07D 213/803; C07D 213/16; C07D 215/04; C07D 401/04; C07D 405/06; C07D 409/04

(52) U.S. Cl. CPC C07D 213/803; C07D 213/16; C07D 215/04; C07D 401/04; C07D 405/06; C07D 409/04

(57) ABSTRACT

A method for preparing a meta-(chiral allyl)-substituted pyridine compound includes: S1, in a glove box filled with nitrogen, adding a boron catalyst, a solvent, pinacol borane, and pyridine to a reaction flask and reacting the mixture under stirring at room temperature to 120° C. for 1-12 hours to obtain dihydropyridine; S2, adding a metal catalyst and a chiral ligand to the reaction flask, stirring the mixture, then adding an alkali and allyl ester, and reacting the mixture at −10° C. to 60° C. for 12-24 hours to obtain a meta-(chiral allyl)-substituted dihydropyridine; and S3, placing the reaction flask in air, reacting the mixture under stirring at room temperature for 1 minute to 12 hours, then performing distillation under reduced pressure to remove the solvent and separation by column chromatography to obtain the meta-(chiral allyl)-substituted pyridine compound.

### CROSS REFERENCE TO THE RELATED APPLICATIONS

This application is the national phase entry of International Application No. PCT/CN2023/100491, filed on Jun. 15, 2023, which is based upon and claims priority to Chinese Patent Application No. 202310414762.5, filed on Apr. 18, 2023, the entire contents of which are incorporated herein by reference.

### TECHNICAL FIELD

The present invention belongs to the field of chemical synthesis and more particularly to a method for preparing a meta-(chiral allyl)-substituted pyridine compound.

### BACKGROUND

A pyridine structure is widely present in natural products, pharmaceuticals, functional materials, and catalytic ligands. In numerous bioactive molecules and catalyst ligands, pyridine is connected to a chiral group (E. Vitaku, D. T. Smith, J. T. Njardarson. J. Med. Chem. 2014, 57, 10257-10274; and P. Bhutani, G. Joshi, N. Raja, N. Bachhav, P. K. Rajanna, H. Bhutani, A. T. Paul, R. Kumar. J. Med. Chem. 2021, 64, 2339-2381). Therefore, the asymmetric functionalization reaction of pyridine represents a significant research direction. At present, it has been reported that the asymmetric alkylation reaction of pyridine in ortho- and para-positions has been realized by strategies such as Minisci reaction and C—H bond activation (R. S. J. Proctor, H. J. Davis, R. J. Phipps. Science 2018, 360, 419-422; J.-F. Li, D. Pan, H.-R. Wang, T. Zhang, Y Li, G. Huang, M.-C. Ye. J. Am. Chem. Soc. 2022, 144, 18810-18816; J.-B. Ma, X. Zhao, D. Zhang, S.-L. Shi. J Am. Chem. Soc. 2022, 144, 13643-13651; and M. W. Jr. Gribble, S. Guo, S. L. Buchwald. J. Am. Chem. Soc. 2018, 140, 5057-5060). However, the asymmetric functionalization reaction of pyridine in the meta-position is very challenging, especially a reaction of converting a C—H bond in the meta-position to chiral allyl has not been reported yet.

### SUMMARY

In order to solve the above problems, the present invention provides a method for preparing a meta-(chiral allyl)-substituted pyridine compound. This method has good functional group compatibility, mild reaction conditions, easy scale-up production, and relatively good chemical selectivity, regioselectivity and enantioselectivity.

To this end, the following technical solution is used in the present invention:

A method for preparing a meta-(chiral allyl)-substituted pyridine compound, including the following steps:
S1, preparation of dihydropyridine:
in a glove box filled with nitrogen, adding pyridine, a catalyst, a solvent, and pinacol borane to a reaction flask and fully reacting the mixture under stirring to obtain 1,4-dihydropyridine or 1,2-dihydropyridine, the reaction equation being as follows:

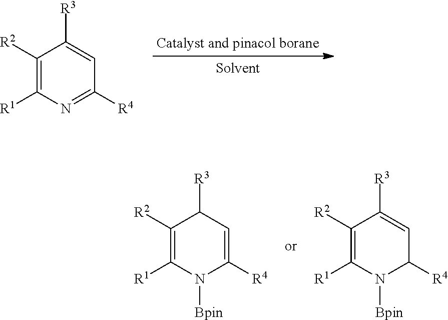

wherein:
the catalyst is triarylboron, the molar ratio of the triarylboron to the pyridine is (2.5-10) 100, and the structural formula of the triarylboron is B(R⁵)₃ in which R⁵ is phenyl, 3,5-bis(trifluoromethyl)-substituted phenyl, or 2,4,6-trifluoro-substituted phenyl;
the equivalent ratio of the pinacol borane to the pyridine is (1-2): 1;
R¹ is hydrogen, alkyl, aryl, alkenyl, substituted aryl, or heteroaryl;
R² is hydrogen, alkyl, halogen, amino, ether group, ester group, aryl, alkenyl, alkynyl, substituted aryl, or heteroaryl;
R³ is hydrogen, alkyl, aryl, or substituted aryl; and
R⁴ is hydrogen, alkyl, aryl, alkenyl, substituted aryl, or heteroaryl;
S2, metal-catalyzed asymmetric allylation reaction of pyridine:
in a glove box filled with nitrogen, adding a metal catalyst, a chiral ligand, and a solvent to a reaction flask, fully stirring the mixture, then adding an alkali, stirring the mixture, then adding an allyl reagent, finally adding the 1,4-dihydropyridine or 1,2-dihydropyridine prepared in step S1, which has been cooled to room temperature, and stirring the mixture until the reaction is complete, to obtain a dihydropyridine substituted with chiral allyl in the meta-position, the reaction equation being as follows:

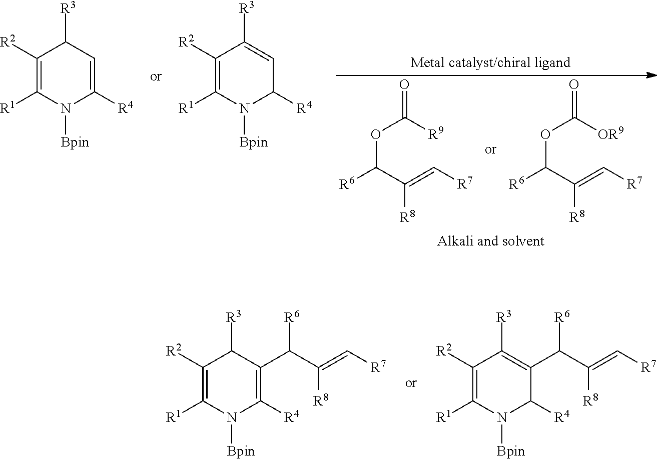

wherein
the metal catalyst is selected from any one of [Ir(COD)Cl]₂, [Ir(COD)OMe]₂, Ir(COD)BF₄, and Pd(P^{t}Bu₃)₂, and the molar ratio of the metal catalyst to the pyridine is (1-10): 100;
the structural formula of the chiral ligand is as follows:

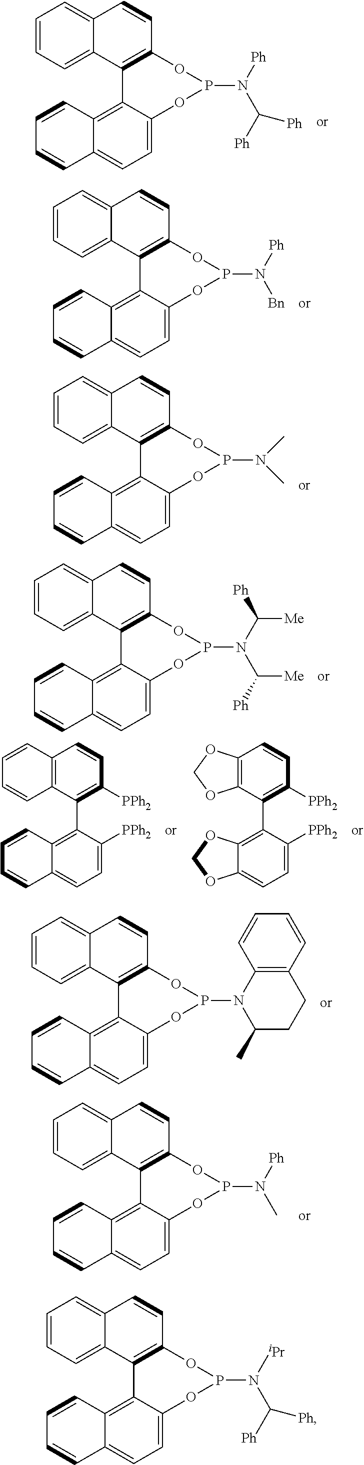

the molar ratio of the chiral ligand to the pyridine is (1-20): 100;
the alkali is triethylamine, diisopropylethylamine, 1,5,7-triazabicyclo[4.4.0]dec-5-ene, 1,5-diazabicyclo[5.4.0]-5-undecene, N-ethylpiperidine, 1,4-diazabicyclo[2.2.2]octane, sodium carbonate, or cesium carbonate, and the equivalent ratio of the alkali to the pyridine is (1-200) 100;
the allyl reagent is allyl carboxylate or allyl carbonate, and the equivalent ratio of the allyl reagent to the pyridine is (1-5): 1;
R⁶ is alkyl, aryl, alkenyl, substituted aryl, or heteroaryl;
R⁷ is hydrogen, alkyl, alkenyl, alkynyl, aryl, substituted aryl, or heteroaryl;
R⁸ is hydrogen or alkyl;
R⁹ is aryl or alkyl; and
the reaction time of the reaction is 16-24 hours; and
S3, oxidative aromatization to obtain a meta-(chiral allyl)-substituted pyridine compound:
placing the reaction flask in step S2 in air, stirring the mixture until the oxidative aromatization reaction is complete, performing distillation under reduced pressure to remove the solvent and then separation by column chromatography to obtain the meta-(chiral allyl)-substituted pyridine compound, the reaction equation being as follows:

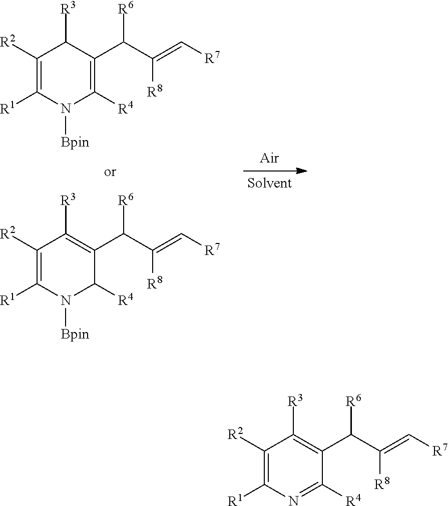

wherein the reaction temperature is room temperature and the reaction time is 1 minute to 24 hours; and
the solvents in steps S1 and S2 are the same and are both selected from at least one of trichloromethane, dichloromethane, 1,2-dichloroethane, ethyl acetate, ethylene glycol dimethyl ether, methyl tert-butyl ether, 1,4-dioxane, methyl cyclopentyl ether, methyl tetrahydrofuran, tetrahydrofuran, toluene, trifluorotoluene, o-xylene, m-xylene, p-xylene, deuterated chloroform, or deuterated tetrahydrofuran.

Preferably, in step S2, the allyl reagent is:

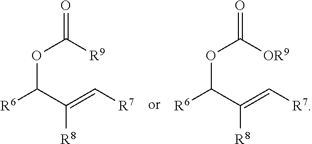

Preferably, the equivalent ratio of the allyl reagent

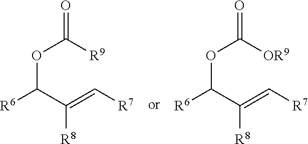

to the pyridine is 1.5:1-3:1.

Preferably, in step S1, the reaction temperature is from room temperature to 120° C., and the reaction time is 1-12 hours; and in step S2, the reaction temperature is −10° C. to 60° C. More preferably, the reaction temperature in step S1 is 40° C.-80° C., and the reaction temperature in step S2 is 30° C.-60° C.

Preferably, in step S1, when R¹ is heteroaryl, the heteroaryl is furyl or thienyl; when R² is heteroaryl, the heteroaryl is furyl, thienyl, benzothiophenyl, benzofuryl, or pyridinyl; and when R⁴ is heteroaryl, the heteroaryl is furyl or thienyl.

Preferably, in step S1, the equivalent ratio of the pinacol borane to the pyridine is 1.5:1.

Preferably, in step S2, when R⁶ is heteroaryl, the heteroaryl is furyl, thienyl, pyridinyl, or indolyl; and when R⁷ is alkyl, the alkyl is propyl, butyl, pentyl, or cyclohexyl. When R⁷ is heteroaryl, the heteroaryl is furyl, thienyl, or pyridinyl. The alkali is preferably 1,4-diazabicyclo[2.2.2]octane, and the preferred equivalent ratio of the alkali to the pyridine is 1:1.

In the present invention, with a boron-based Lewis acid and metal iridium or palladium as a catalyst, a de-aromatization-re-aromatization strategy of pyridine is used to realize the asymmetric allylation reaction of pyridine in the meta-position, wherein firstly, under the action of catalysis of the boron-based Lewis acid, the pyridine reacts with pinacol borane to generate electron-rich 1,4-dihydropyridine or 1,2-dihydropyridine; in the second step, an asymmetric allylation reaction of the dihydropyridine occurs; and finally, oxidative aromatization occurs to obtain the meta-(chiral allyl)-substituted pyridine product.

Compared with the prior art, the present invention has the following beneficial effects:
1. by using a novel boron/metal co-catalysis system, the present invention directly converts the C—H bond in the meta-position of pyridine into chiral allyl for the first time and achieves relatively good step economy and atomic economy;
2. the pyridine and allyl ester used in the present invention are both commercially available chemicals, which are beneficial to industrial scale-up production;
3. in the present invention, the asymmetric allylation reaction only occurs in the meta-position of pyridine, but not in the ortho-position and para-position of pyridine and other aromatic rings, achieving relatively good chemical selectivity and regioselectivity;
4. the reaction of the present invention takes place under relatively mild conditions, and the product can be obtained by the reaction even at −10° C. to 120° C.;
5. the product obtained by the present invention is easily further converted into derivatives such as 3-(chiral alkyl)pyridine and 3-(chiral hydroxyalkyl)pyridine, so it has a wide application prospect in medicine, ligand development, etc.;
6. the present invention can be used for the late functional modification of drug molecules containing pyridine structure and can be applied to the research and for the development and production of drugs; and
7. during the reaction process of the present invention, only 1 equivalent of the pyridine raw material is required, and the pyridine raw material does not need to be used as a solvent or in excess, so that the atom utilization rate is high.

### DETAILED DESCRIPTION OF THE EMBODIMENTS

The method of the present invention will be described in detail below with respect to specific examples.

In the reaction equations of the following examples, LA1, LA2, and LA3 are catalysts; L1, L2, L3, and L4 are chiral ligands; HBpin is pinacol borane; THF is tetrahydrofuran; DABCO is 1,4-diazabicyclo[2.2.2]octane; and equiv is equivalent.

### Example 1

A method for preparing (R)-3-phenyl-5-(1-phenylallyl)pyridine, including the following steps:
S1, hydroboration reaction to prepare dihydropyridine: in a glove box filled with nitrogen, 4.0 mg of the catalyst B(2,4,6-F₃C₆H₂)₃ (LA1) (0.01 mmol, 5.0 mol %), 0.5 mL of tetrahydrofuran, 38.4 mg of pinacol borane (0.30 mmol, 1.5 equiv), and 31.0 mg of 3-phenylpyridine (1a) (0.2 mmol, 1.0 equiv) were added to an 8 mL reaction flask, stirred, and reacted at 80° C. for 12 hours to obtain 3-phenyl-1-(4,4,5,5-tetramethyl-1,3,2-dioxaborolane)-1,4-dihydropyridine, the reaction equation being as follows:

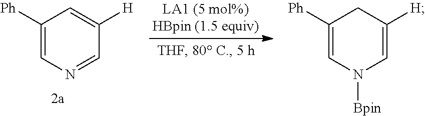

in which the structural formula of the catalyst LA1 was as follows:

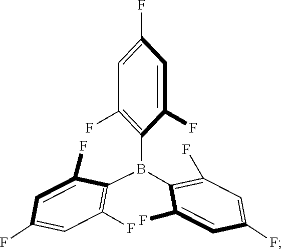

S2, iridium-catalyzed asymmetric allylation reaction of dihydropyridine: in a glove box filled with nitrogen, 3.4 mg of [Ir(COD)Cl]₂ (0.005 mmol, 2.5 mol %), 6.3 mg of the chiral ligand L1 (0.011 mmol, 5.5 mol %), and 0.5 mL of the solvent tetrahydrofuran were added to a reaction flask and fully stirred; 22.4 mg of 1,4-diazabicyclo[2.2.2]octane (0.2 mmol, 1.0 equiv) was then added and stirred; 130.8 mg of 1-phenylallyl pivalate (0.6 mmol, 3.0 equiv) was then added; and finally, 1,4-dihydropyridine cooled to room temperature was added and stirred for 24 hours to obtain (R)-1-(4,4,5,5-tetramethyl-1,3,2-dioxaborolane)-3-phenyl-5-(1-phenylallyl)-1,4-dihydropyridine, which was substituted with allyl in the meta-position, the reaction equation being as follows:

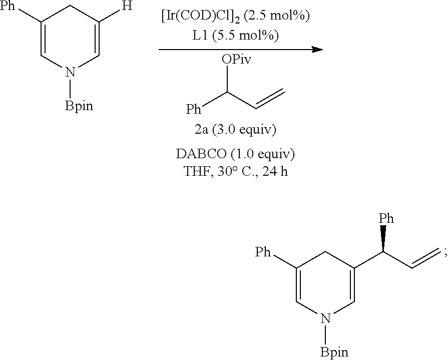

in which the structural formula of the ligand L1 was as follows:

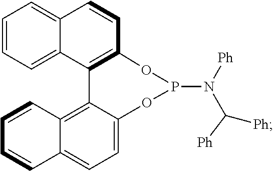

and
S3, oxidative aromatization to obtain a meta-allyl-substituted pyridine compound: the system was cooled to room temperature and stirred in air for 6 hours to generate (R)-3-phenyl-5-(1-phenylallyl)pyridine (3a), the reaction equation being as follows:

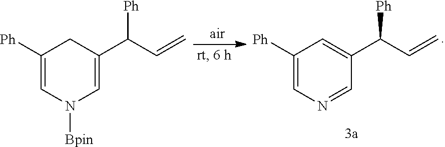

After the reaction was complete, the solvent was removed by rotary evaporation, and the residue was separated and purified by silica gel column chromatography to obtain the product (R)-3-phenyl-5-(1-phenylallyl)pyridine as a colorless liquid, with a yield of 75% and an ee value of 98%.

The product was characterized as follows: [α]²⁰_{D}=−2.4 (c=0.38, CHCl₃). ¹H NMR (400 MHz, CDCl₃) δ 8.71 (d, J=2.2 Hz, 1H), 8.46 (d, J=2.2 Hz, 1H), 7.66 (t, J=2.2 Hz, 1H), 7.54-7.50 (m, 2H), 7.46-7.40 (m, 2H), 7.39-7.29 (m, 3H), 7.28-7.20 (m, 3H), 6.38-6.27 (m, 1H), 5.30 (d, J=10.2 Hz, 1H), 5.04 (d, J=17.0 Hz, 1H), 4.82 (d, J=7.0 Hz, 1H). ¹³C NMR (101 MHz, CDCl₃) δ 150.9, 146.5, 142.0, 139.6, 138.7, 137.9, 136.4, 134.5, 129.1, 128.8, 128.6, 128.2, 127.3, 126.9, 117.5, 52.6. HRMS-ESI: m/z calculated for C₂₀H₁₈N⁺ (M+H)⁺ 272.1434, found: 272.1434. HPLC retention times (AD-H, eluent: hexanes/i-PrOH=95/5, 250 nm, flow rate: 0.5 mL/min, T=25° C.): t_{R}=26.9 min (major), t_{R}=30.0 min (minor).

### Example 2

A method for preparing (R)-3-(1-phenylallyl)-5,6,7,8-tetrahydroquinoline, including the following steps:
S1, in a glove box filled with nitrogen, 6.5 mg of the catalyst (LA2) (0.01 mmol, 5.0 mol %), 0.5 mL of tetrahydrofuran, 38.4 mg of pinacol borane (0.30 mmol, 1.5 equiv), and 26.6 mg of 5,6,7,8-tetrahydroquinoline (1b) (0.2 mmol, 1.0 equiv) were added to an 8 mL small reaction flask, stirred, and reacted at 80° C. for 12 hours to obtain 1-(4,4,5,5-tetramethyl-1,3,2-dioxaborolane)-1,4,5,6,7,8-hexahydroquinoline, the reaction equation being as follows:

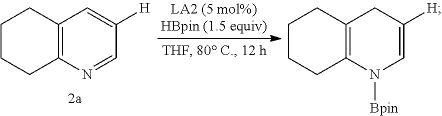

in which the structural formula of the catalyst LA2 was as follows:

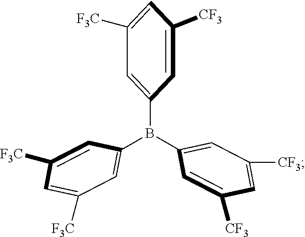

S2, iridium-catalyzed asymmetric allylation reaction of dihydropyridine: in a glove box filled with nitrogen, 3.4 mg of [Ir(COD)Cl]₂ (0.005 mmol, 2.5 mol %), 6.3 mg of the ligand L1 (0.011 mmol, 5.5 mol %), and 0.5 mL of the solvent tetrahydrofuran were added to a reaction flask and fully stirred; 22.4 mg of 1,4-diazabicyclo[2.2.2]octane (0.2 mmol, 1.0 equiv) was then added and stirred; 130.8 mg of 1-phenylallyl pivalate (0.6 mmol, 3.0 equiv) was then added; and finally, a 1,4-dihydropyridine solution cooled to room temperature was added and stirred at 30° C. for 24 hours to obtain (R)-3-(1-phenylallyl)-1-(4,4,5,5-tetramethyl-1,3,2-dioxaborolane)-1,4,5,6,7,8-hexahydroquinolin e, which was substituted with allyl in the meta-position, the reaction equation being as follows:

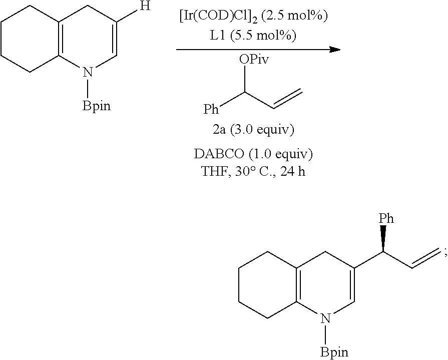

in which the structural formula of the ligand L1 was as follows:

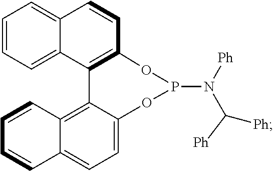

and
S3, oxidative aromatization to obtain a meta-allyl-substituted pyridine compound:
the system was cooled to room temperature and stirred in air for 6 hours to generate (R)-3-(1-phenylallyl)-5,6,7,8-tetrahydroquinoline (3b), the reaction equation being as follows:

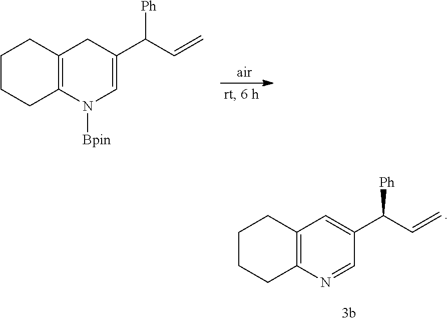

After the reaction was complete, the solvent was removed by rotary evaporation, and the residue was separated and purified by silica gel column chromatography to obtain the product (R)-3-(1-phenylallyl)-5,6,7,8-tetrahydroquinoline as a colorless liquid, with a yield of 48% and an ee value >99%.

The product was characterized as follows:
[α]²⁰_{D}=+8.9 (c=0.27, CHCl₃). ¹H NMR (400 MHz, CDCl₃) δ 8.21 (d, J=2.2 Hz, 1H), 7.34-7.16 (m, 5H), 7.12 (d, J=2.2 Hz, 1H), 6.33-6.21 (m, 1H), 5.25 (dt, J=10.2, 1.4 Hz, 1H), 5.00 (dt, J=17.1, 1.4 Hz, 1H), 4.68 (d, J=7.1 Hz, 1H), 2.89 (t, J=6.4 Hz, 2H), 2.70 (t, J=6.3 Hz, 2H), 1.93-1.83 (m, 2H), 1.83-1.72 (m, 2H). ¹³C NMR (101 MHz, CDCl₃) δ 155.6, 147.2, 142.6, 140.0, 136.8, 135.8, 132.0, 128.7, 128.6, 126.7, 117.0, 52.3, 32.3, 28.9, 23.2, 22.8. HRMS-ESI: m/z calculated for C₁₈H₂₀N⁺ (M+H)⁺ 250.1590, found: 250.1589. HPLC retention times (AD-H, eluent: hexanes/i-PrOH=95/5, 254 nm, flow rate: 0.5 mL/min, T=25° C.): t_{R}=15.0 min (major), t_{R}=17.3 min (minor).

### Example 3

A method for preparing (R)-2-phenyl-3-(1-phenylallyl)pyridine and 2-phenyl-3,5-bis((R)-1-phenylallyl)pyridine, including the following steps:
S1, in a glove box filled with nitrogen, 13.0 mg of the catalyst (LA2) (0.02 mmol, 5.0 mol %), 0.5 mL of tetrahydrofuran, 76.8 mg of pinacol borane (0.60 mmol, 1.5 equiv), and 62.0 mg of 2-phenylpyridine (1c) (0.4 mmol, 1.0 equiv) were added to an 8 mL small reaction flask, stirred, and reacted at 40° C. for 3 hours to obtain 2-phenyl-1-(4,4,5,5-tetramethyl-1,3,2-dioxaborolane)-1,4-dihydropyridine and 2-phenyl-1-(4,4,5,5-tetramethyl-1,3,2-dioxaborolane)-1,6-dihydropyridine, the reaction equation being as follows:

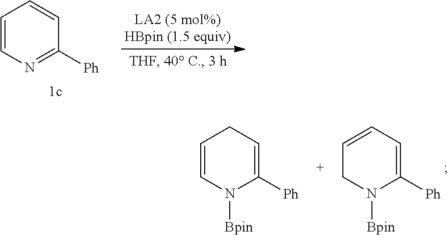

in which the structural formula of the catalyst LA2 was as follows:

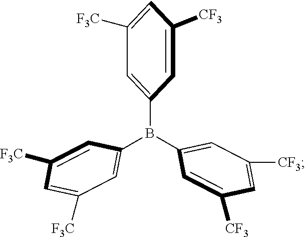

S2, iridium-catalyzed asymmetric allylation reaction of dihydropyridine: in a glove box filled with nitrogen, 6.8 mg of [Ir(COD)Cl]₂ (0.01 mmol, 2.5 mol %), 12.6 mg of the ligand L1 (0.022 mmol, 5.5 mol %), 0.5 mL of the solvent tetrahydrofuran were added to a reaction flask and fully stirred; 44.8 mg of 1,4-diazabicyclo[2.2.2]octane (0.4 mmol, 1.0 equiv) was added and then stirred; 348.8 mg of 1-phenylallyl pivalate (1.6 mmol, 4.0 equiv) was added; and finally, a mixed solution of 1,4-dihydropyridine and 1,6-dihydropyridine cooled to room temperature was added was added and stirred at 30° C. for 24 hours to obtain (R)-1-(4,4,5,5-tetramethyl-1,3,2-dioxaborolane)-2-phenyl-3-(1-phenylallyl)-1,4-dihydropyridine and 1-(4,4,5,5-tetramethyl-1,3,2-dioxaborolane)-2-phenyl-3,5-bis((R)-1-phenylallyl)-1,6-dihydropyri dine, which were substituted with allyl in the meta-position, the reaction equation being as follows:

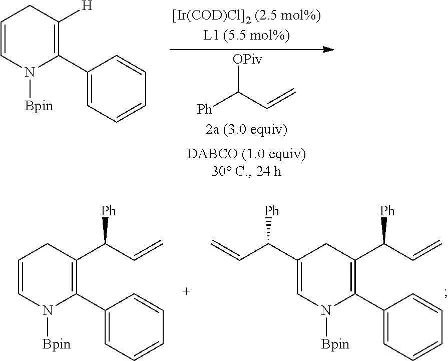

in which the structural formula of the ligand L1 was as follows:

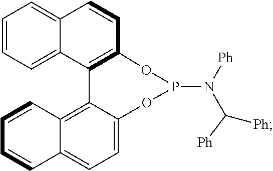

and
S3, oxidative aromatization to obtain a C3 allyl-substituted pyridine compound and C3 and C5 diallyl-substituted pyridine compound: the system was cooled to room temperature and stirred in air for 6 hours to generate (R)-2-phenyl-3-(1-phenylallyl)pyridine (3c) and 2-phenyl-3,5-bis((R)-1-phenylallyl)pyridine (3c′), the reaction equation being as follows:

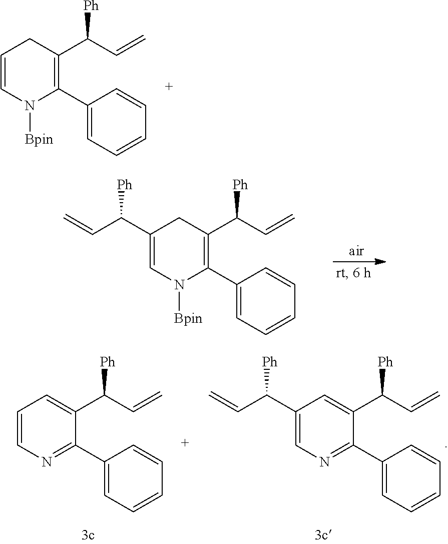

After the reaction was complete, the solvent was removed by rotary evaporation, and the residue was separated and purified by silica gel column chromatography to obtain the products (R)-2-phenyl-3-(1-phenylallyl)pyridine and 2-phenyl-3,5-bis((R)-1-phenylallyl)pyridine, wherein the (R)-2-phenyl-3-(1-phenylallyl)pyridine was a colorless liquid, with a yield of 44% and an ee value of 99%; and the 2-phenyl-3,5-bis((R)-1-phenylallyl)pyridine was a colorless liquid, with a yield of 30%, ee value >99%, and dr value >30:1.

The products were characterized as follows:
(R)-2-phenyl-3-(1-phenylallyl)pyridine: [α]²⁰_{D}=+129.3 (c=0.93, CHCl₃). ¹H NMR (400 MHz, CDCl₃) δ 8.56 (dd, J=4.7, 1.7 Hz, 1H), 7.56 (dd, J=7.9, 1.7 Hz, 1H), 7.47-7.38 (m, 5H), 7.28-7.16 (m, 4H), 7.08-7.03 (m, 2H), 6.28-6.16 (m, 1H), 5.28 (dt, J=10.2, 1.4 Hz, 1H), 4.96 (d, J=6.2 Hz, 1H), 4.84 (dt, J=17.2, 1.4 Hz, 1H). ¹³C NMR (101 MHz, CDCl₃) δ 159.3, 147.5, 142.8, 140.4, 140.4, 137.7, 136.3, 129.0, 128.6, 128.6, 128.3, 128.2, 126.6, 122.5, 117.6, 50.1. HRMS-ESI: m/z calculated for C₂₀H₁₈N⁺ (M+H)⁺ 272.1434, found: 272.1428. HPLC retention times (AD-H, eluent: hexanes/i-PrOH=98/2, 250 nm, flow rate: 0.5 mL/min, T=25° C.): t_{R}=14.5 min (major), t_{R}=17.0 min (minor).

2-Phenyl-3,5-bis((R)-1-phenylallyl)pyridine: [α]²⁰_{D}=+39.9 (c=0.63, CHCl₃). ¹H NMR (400 MHz, CDCl₃) δ 8.39 (d, J=2.2 Hz, 1H), 7.44-7.32 (m, 6H), 7.34-7.28 (m, 2H), 7.26-7.14 (m, 6H), 6.99-6.96 (m, 2H), 6.39-6.08 (m, 2H), 5.32-5.19 (m, 2H), 5.05-4.88 (m, 2H), 4.85-4.70 (m, 2H). ¹³C NMR (101 MHz, CDCl₃) δ 157.4, 147.7, 142.7, 142.2, 140.4, 140.1, 139.6, 137.8, 137.3, 135.8, 129.1, 128.7, 128.6, 128.5, 128.5, 128.2, 128.1, 126.9, 126.6, 117.4, 117.3, 51.5, 50.1. HRMS-ESI: m/z calculated for C₂₉H₂₆N⁺ (M+H)⁺ 388.2060, found: 388.2065. HPLC retention times (AD-H, eluent: hexanes/i-PrOH=98/2, 254 nm, flow rate: 0.5 mL/min, T=25° C.): t_{R}=16.2 min (minor), t_{R}=17.6 min (major), t_{R}=21.3 min (minor), t_{R}=28.1 min (minor).

### Example 4

A method for preparing (R)-3-(1-phenylallyl)-2-(2-thienyl)pyridine, including the following steps:
S1, in a glove box filled with nitrogen, 13.0 mg of the catalyst (LA2) (0.02 mmol, 5.0 mol %), 0.5 mL of tetrahydrofuran, 76.8 mg of pinacol borane (0.60 mmol, 1.5 equiv), and 32.2 mg of 2-(2-thienyl)pyridine (1d) (0.2 mmol, 1.0 equiv) were added to an 8 mL small reaction flask, stirred, and reacted at 40° C. for 3 hours to obtain 2-(2-thienyl)-1-(4,4,5,5-tetramethyl-1,3,2-dioxaborolane)-1,4-dihydropyridine and 2-(2-thienyl)-1-(4,4,5,5-tetramethyl-1,3,2-dioxaborolane)-1,6-dihydropyridine, the reaction equation being as follows:

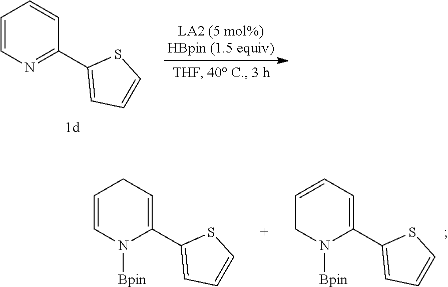

in which the structural formula of the catalyst LA2 was as follows:

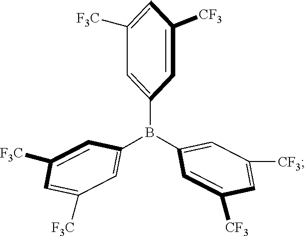

S2, iridium-catalyzed asymmetric allylation reaction of dihydropyridine: in a glove box filled with nitrogen, 6.8 mg of [Ir(COD)Cl]₂ (0.01 mmol, 2.5 mol %), 12.6 mg of the ligand L1 (0.022 mmol, 5.5 mol %), and 0.5 mL of the solvent tetrahydrofuran were added to a reaction flask and fully stirred; 44.8 mg of 1,4-diazabicyclo[2.2.2]octane (0.4 mmol, 1.0 equiv) was then added and stirred; 348.8 mg of 1-phenylallyl pivalate (1.6 mmol, 4.0 equiv) was then added; and finally, a mixed solution of 1,4-dihydropyridine and 1,6-dihydropyridine cooled to room temperature was added and stirred at 30° C. for 24 hours to obtain (R)-1-(4,4,5,5-tetramethyl-1,3,2-dioxaborolane)-2-(2-thienyl)-3-(1-phenylallyl)-1,4-dihydropyrid ine, which was substituted with allyl in the meta-position, the reaction equation being as follows:

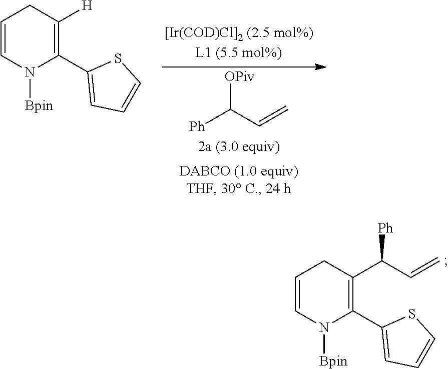

S3, the system was stirred at room temperature in air for 6 hours to generate (R)-3-(1-phenylallyl)-2-thiophen-2-yl)pyridine (3d), the reaction equation being as follows:

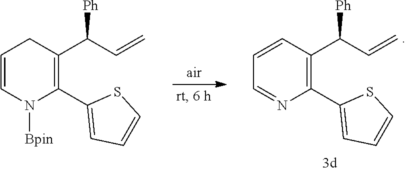

After the reaction was complete, the solvent was removed by rotary evaporation, and the residue was separated and purified by silica gel column chromatography to obtain the product (R)-3-(1-phenylallyl)-2-(2-thienyl)pyridine as a colorless liquid, with a yield of 40% and an ee value of 99%.

The product was characterized as follows:
[α]²⁰_{D}=115.67 (c=0.8, CHCl₃). ¹H NMR (400 MHz, CDCl₃) δ 8.53 (d, J=4.7 Hz, 1H), 7.52 (d, J=9.7 Hz, 1H), 7.41 (d, J=5.1 Hz, 1H), 7.35-7.26 (m, 2H), 7.25-7.22 (m, 2H), 7.19-7.10 (m, 3H), 7.06-7.01 (m, 1H), 6.35-6.25 (m, 1H), 5.35-5.29 (m, 2H), 4.87 (d, J=18.9 Hz, 1H). ¹³C NMR (101 MHz, CDCl₃) δ 150.8, 147.5, 143.3, 141.8, 140.2, 140.2, 138.5, 135.3, 128.9, 128.7, 127.6, 127.4, 127.4, 126.8, 122.2, 118.0, 49.6. HRMS-ESI: m/z calculated for C₁₈H₁₆NS⁺ (M+H)⁺ 278.0998, found: 278.0998. HPLC retention times (AD-H, eluent: hexanes/i-PrOH=98/2, 230 nm, flow rate: 0.5 mL/min, T=25° C.): t_{R}=14.2 min (major), t_{R}=15.5 min (minor).

### Example 5

A method for preparing (R)-3-butyl-5-(1-phenylallyl)pyridine, including the following steps:
S1, in a glove box filled with nitrogen, 2.4 mg of the catalyst (LA3) (0.01 mmol, 5.0 mol %), 0.5 mL of tetrahydrofuran, 38.4 mg of pinacol borane (0.30 mmol, 1.5 equiv), and 27.0 mg of 3-butylpyridine (5a) (0.2 mmol, 1.0 equiv) were added to an 8 mL small reaction flask, stirred, and reacted at 80° C. for 12 hours to obtain 1-(4,4,5,5-tetramethyl-1,3,2-dioxaborolane)-3-butyl-1,4-dihydropyridine, the reaction equation being as follows:

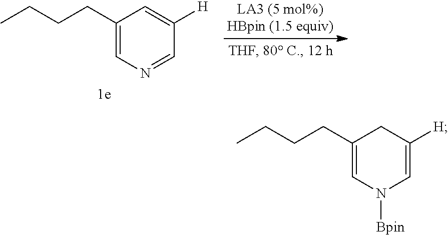

in which the structural formula of the catalyst LA3 was as follows:

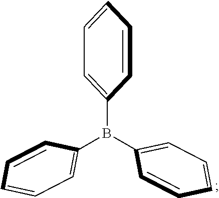

S2, iridium-catalyzed asymmetric allylation reaction of dihydropyridine: in a glove box filled with nitrogen, 5.0 mg of Ir(COD)BF₄ (0.01 mmol, 5 mol %), 6.3 mg of the ligand L1 (0.011 mmol, 5.5 mol %), and 0.5 mL of the solvent tetrahydrofuran were added to a reaction flask and fully stirred; 20.2 mg of triethylamine (0.2 mmol, 1.0 equiv) was then added and stirred; 130.8 mg of 1-phenylallyl pivalate (0.6 mmol, 3.0 equiv) was then added; and finally, a 1,4-dihydropyridine solution cooled to room temperature was added and stirred at 30° C. for 24 hours to obtain (R)-3-butyl-1-(4,4,5,5-tetramethyl-1,3,2-dioxaborolane)-5-(1-phenylallyl)-1,4-dihydropyridine, which was substituted with allyl in the meta-position, the reaction equation being as follows:

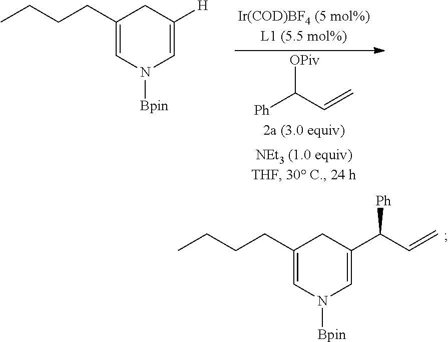

in which the structural formula of the ligand L1 was as follows:

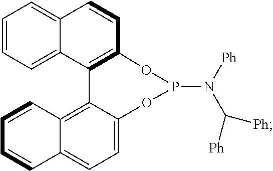

and
S3, oxidative aromatization to obtain a meta-allyl-substituted pyridine compound: the system was cooled to room temperature and stirred in air for 6 hours to generate (R)-3-butyl-5-(1-phenylallyl)pyridine (3e), the reaction equation being as follows:

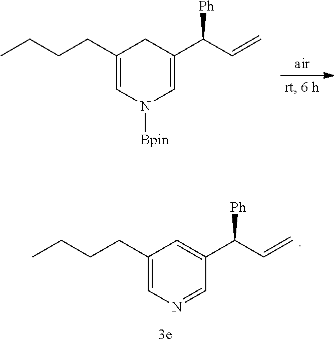

After the reaction was complete, the solvent was removed by rotary evaporation, and the residue was separated and purified by silica gel column chromatography to obtain the product (R)-3-butyl-5-(1-phenylallyl)pyridine as a colorless liquid, with a yield of 42% and an ee value of 96%.

The product was characterized as follows:
[α]²⁰_{D}=−3.4 (c=0.43, CHCl₃). ¹H NMR (400 MHz, CDCl₃) δ 8.31-8.20 (m, 2H), 7.34-7.20 (m, 4H), 7.20-7.14 (m, 2H), 6.35-6.20 (m, 1H), 5.34-5.18 (m, 1H), 5.00 (dt, J=17.1, 1.5 Hz, 1H), 4.72 (d, J=7.0 Hz, 1H), 2.56 (t, J=8.0, 2H), 1.63-1.49 (m, 2H), 1.42-1.26 (m, 2H), 0.91 (t, J=7.3 Hz, 3H). ¹³C NMR (101 MHz, CDCl₃) δ 148.1, 147.6, 142.3, 139.8, 138.2, 137.8, 136.0, 128.7, 128.6, 126.8, 117.2, 50.7, 33.4, 32.8, 25.9, 13.1. HRMS-ESI: m/z calculated for C₁₈H₂₂N⁺ (M+H)⁺ 252.1747, found: 224.1753. HPLC retention times (AD-H, eluent: hexanes/i-PrOH=95/5, 254 nm, flow rate: 0.5 mL/min, T=25° C.): t_{R}=16.3 min (major), t_{R}=17.3 min (minor).

### Example 6

A method for preparing (R)-2-methyl-5-phenyl-3-(1-phenylallyl)pyridine, including the following steps:
S1, in a glove box filled with nitrogen, 6.5 mg of the catalyst (LA2) (0.01 mmol, 5.0 mol %), 0.5 mL of tetrahydrofuran, 38.4 mg of pinacol borane (0.30 mmol, 1.5 equiv), and 33.8 mg of 2-methyl-5-phenylpyridine (if) (0.2 mmol, 1.0 equiv) were added to an 8 mL small reaction flask, stirred, and reacted at 80° C. for 12 hours to obtain 2-methyl-5-phenyl-1-(4,4,5,5-tetramethyl-1,3,2-dioxaborolane)-1,4-dihydropyridine, the reaction equation being as follows:

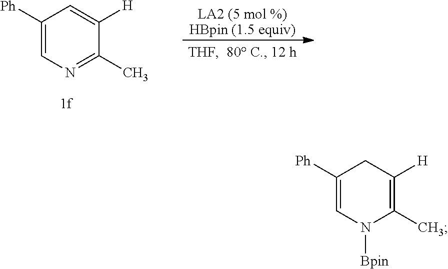

in which the structural formula of the catalyst LA2 was as follows:

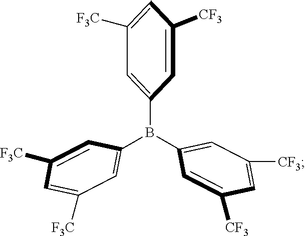

S2, iridium-catalyzed asymmetric allylation reaction of dihydropyridine: in a glove box filled with nitrogen, 3.4 mg of [Ir(COD)Cl]₂ (0.005 mmol, 2.5 mol %), 6.3 mg of the ligand L1 (0.011 mmol, 5.5 mol %), and 0.5 mL of the solvent tetrahydrofuran were added to a reaction flask and fully stirred; 22.4 mg of 1,4-diazabicyclo[2.2.2]octane (0.2 mmol, 1.0 equiv) was then added and stirred; 130.8 mg of 1-phenylallyl pivalate (0.6 mmol, 3.0 equiv) was then added; and finally, a 1,4-dihydropyridine solution cooled to room temperature was added and stirred at 30° C. for 24 hours to obtain (R)-2-methyl-5-phenyl-3-(1-phenylallyl)-1-(4,4,5,5-tetramethyl-1,3,2-dioxaborolane)-1,4-dihydr opyridine, which was substituted with allyl in the meta-position, the reaction equation being as follows:

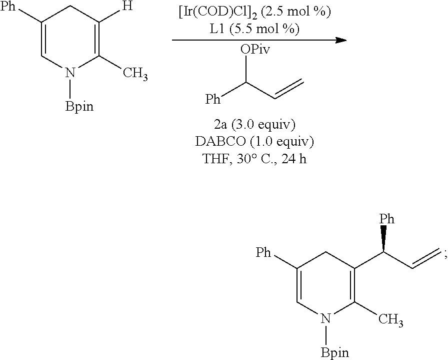

in which the structural formula of the ligand L1 was as follows:

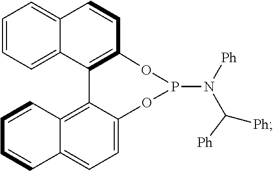

and
S3, oxidative aromatization to obtain a meta-allyl-substituted pyridine compound: the system was cooled to room temperature and stirred in air for 6 hours to generate (R)-2-methyl-5-phenyl-3-(1-phenylallyl) pyridine (3f), the reaction equation being as follows:

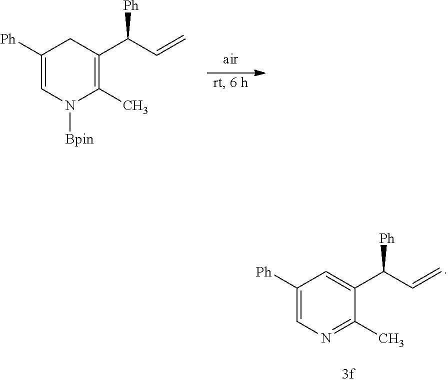

After the reaction was complete, the solvent was removed by rotary evaporation, and the residue was separated and purified by silica gel column chromatography to obtain the product (R)-2-methyl-5-phenyl-3-(1-phenylallyl)pyridine as a yellow liquid, with a yield of 65% and an ee value of 97%.

The product was characterized as follows:
[α]²⁰_{D}=−128.4 (c=0.97, CHCl₃). ¹H NMR (400 MHz, CDCl₃) δ 8.62 (d, J=2.3 Hz, 1H), 7.62 (d, J=2.3 Hz, 1H), 7.54-7.49 (m, 2H), 7.46-7.42 (m, 2H), 7.39-7.29 (m, 3H), 7.28-7.23 (m, 1H), 7.18-7.13 (m, 2H), 6.35-6.25 (m, 1H), 5.30 (d, J=10.0 Hz, 1H), 5.06-4.83 (m, 2H), 2.53 (s, 3H). ¹³C NMR (101 MHz, CDCl₃) δ 156.1, 145.4, 141.3, 139.4, 138.2, 136.6, 134.6, 134.4, 129.1, 128.9, 128.7, 127.9, 127.2, 126.9, 117.6, 50.9, 22.4. HRMS-ESI: m/z calculated for C₂₁H₂₀N⁺ (M+H)⁺ 286.1590, found: 286.1593. HPLC retention times (AD-H, eluent: hexanes/i-PrOH=90/10, 254 nm, flow rate: 0.5 mL/min, T=25° C.): t_{R}=12.1 min (major), t_{R}=15.0 min (minor).

### Example 7

A method for preparing (R)-3-(1-phenylallyl)-4-trifluoromethylpyridine, including the following steps:
S1, in a glove box filled with nitrogen, 6.5 mg of the catalyst (LA2) (0.01 mmol, 5.0 mol %), 0.5 mL of tetrahydrofuran, 38.4 mg of pinacol borane (0.30 mmol, 1.5 equiv), and 29.4 mg of 4-trifluoromethylpyridine (1g) (0.2 mmol, 1.0 equiv) were added to an 8 mL small reaction flask, stirred, and reacted at 80° C. for 12 hours to obtain 4-trifluoromethyl-1-(4,4,5,5-tetramethyl-1,3,2-dioxaborolane)-1,2-dihydropyridine, the reaction equation being as follows:

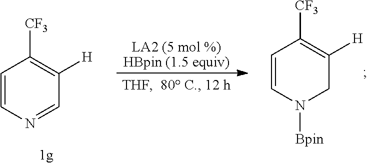

in which the structural formula of the catalyst LA2 was as follows:

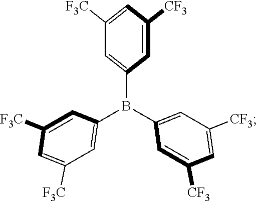

S2, iridium-catalyzed asymmetric allylation reaction of dihydropyridine: in a glove box filled with nitrogen, 6.8 mg of [Ir(COD)Cl]₂ (0.01 mmol, 5 mol %), 12.6 mg of the ligand L1 (0.022 mmol, 11 mol %), and 0.5 mL of the solvent tetrahydrofuran were added to a reaction flask and fully stirred; 22.4 mg of 1,4-diazabicyclo[2.2.2]octane (0.2 mmol, 1.0 equiv) was then added and stirred; 105.7 mg of 1-phenylallyl acetate (0.6 mmol, 3.0 equiv) was then added; and finally, a 1,2-dihydropyridine solution cooled to room temperature was added and stirred at 30° C. for 24 hours to obtain (R)-3-(1-phenylallyl)-4-trifluoromethyl-1-(4,4,5,5-tetramethyl-1,3,2-dioxaborolane)-1,2-dihydro pyridine, which was substituted with allyl in the meta-position, the reaction equation being as follows:

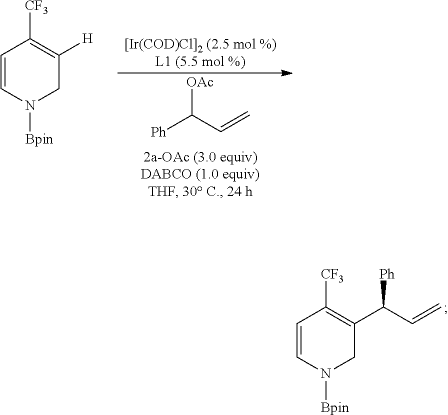

in which the structural formula of the ligand L1 was as follows:

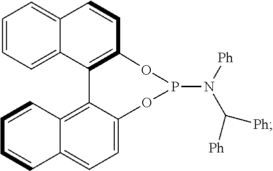

and
S3, oxidative aromatization to obtain a meta-allyl-substituted pyridine compound: the system was cooled to room temperature and stirred in air for 6 hours to generate (R)-3-(1-phenylallyl)-4-(trifluoromethyl)pyridine (3g), the reaction equation being as follows:

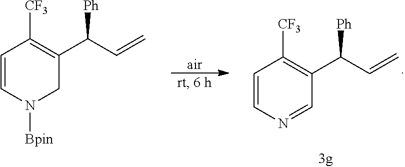

After the reaction was complete, the solvent was removed by rotary evaporation, and the residue was separated and purified by silica gel column chromatography to obtain the product (R)-3-(1-phenylallyl)-4-(trifluoromethyl)pyridine as a yellow liquid, with a yield of 38% and an ee value of 99%.

The product was characterized as follows:
[α]²⁰_{D}=+91.5 (c=0.94, CHCl₃). ¹H NMR (400 MHz, CDCl₃) δ 8.63-8.61 (m, 2H), 7.53-7.50 (m, 1H), 7.33-7.28 (m, 2H), 7.30-7.18 (m, 3H), 6.35-6.26 (m, 1H), 5.32 (d, J=10.2 Hz, 1H), 5.19 (d, J=6.5 Hz, 1H), 4.93 (d, J=17.1 Hz, 1H). ¹³C NMR (101 MHz, CDCl₃) δ 153.4, 148.4, 141.4, 138.9, 136.3, 136.0 (q, J=31.6 Hz), 128.8, 128.5, 127.1, 123.4 (q, J=275.4 Hz), 119.4 (q, J=5.4 Hz), 118.3, 48.0. ¹⁹F NMR (376 MHz, CDCl₃) δ-60.8. HRMS-ESI: m/z calculated for C₁₅H₁₃F₃N⁺ (M+H)⁺ 264.0995, found: 264.0996. HPLC retention times (OJ-H, eluent: hexanes/i-PrOH=95/5, 254 nm, flow rate: 0.5 mL/min, T=25° C.): t_{R}=9.2 min (minor), t_{R}=11.6 min (major).

### Example 8

A method for preparing (R)-3-(1-phenylallyl)quinoline, including the following steps:
S1, in a glove box filled with nitrogen, 6.5 mg of the catalyst (LA1) (0.01 mmol, 5.0 mol %), 0.5 mL of tetrahydrofuran, 38.4 mg of pinacol borane (0.30 mmol, 1.5 equiv), and 25.8 mg of quinoline (li) (0.2 mmol, 1.0 equiv) were added to an 8 mL small reaction flask, stirred, and reacted at 80° C. for 12 hours to obtain 1-(4,4,5,5-tetramethyl-1,3,2-dioxaborolane)-1,4-dihydroquinoline, the reaction equation being as follows:

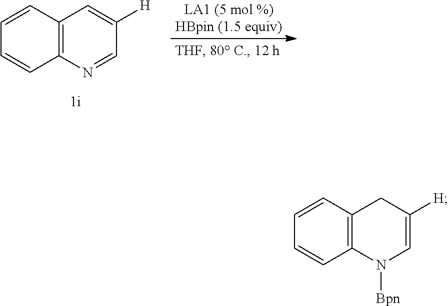

in which the structural formula of the catalyst LA1 was as follows:

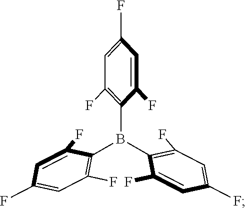

S2, iridium-catalyzed asymmetric allylation reaction of dihydropyridine: 3.4 mg of [Ir(COD)Cl]₂ (0.005 mmol, 2.5 mol %), 6.3 mg of the ligand L1 (0.011 mmol, 5.5 mol %), and 0.5 mL of the solvent tetrahydrofuran were added to a reaction flask and fully stirred; 22.4 mg of 1,4-diazabicyclo[2.2.2]octane (0.2 mmol, 1.0 equiv) was then added and stirred; 130.8 mg of 1-phenylallyl pivalate (0.6 mmol, 3.0 equiv) was then added; and finally, a 1,4-dihydroquinoline solution cooled to room temperature was added and stirred at 30° C. in a nitrogen atmosphere for 24 hours to obtain (R)-3-(1-phenylallyl)-1-(4,4,5,5-tetramethyl-1,3,2-dioxaborolane)-1,4-dihydroquinoline, which was substituted with allyl in the meta-position, the reaction equation being as follows:

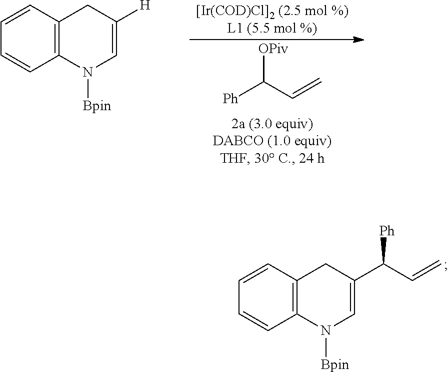

in which the structural formula of the ligand L1 was as follows:

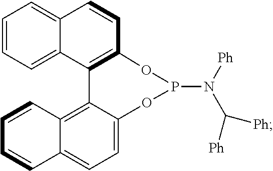

and
S3, oxidative aromatization to obtain a meta-allyl-substituted pyridine compound: the system was cooled to room temperature and stirred in air for 6 hours to generate (R)-3-(1-phenylallyl)quinoline (3i), the reaction equation being as follows:

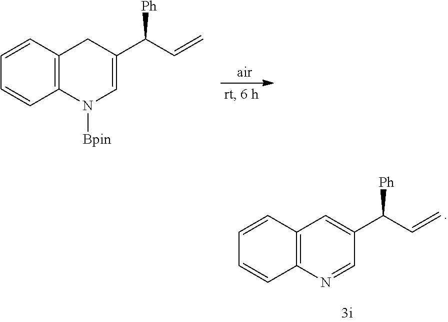

After the reaction was complete, the solvent was removed by rotary evaporation, and the residue was separated and purified by silica gel column chromatography to obtain the product (R)-3-(1-phenylallyl)quinoline as a colorless liquid, with a yield of 49% and an ee value of 97%.

The product was characterized as follows:
[α]²⁰_{D}=−22.1 (c=0.6, CHCl₃). ¹H NMR (400 MHz, CDCl₃) δ 8.78 (d, J=2.2 Hz, 1H), 8.08 (d, J=8.4, 1H), 7.88 (d, J=2.2, 1H), 7.75 (d, J=8.2, 1H), 7.70-7.62 (m, 1H), 7.54-7.50 (m, 1H), 7.38-7.29 (m, 2H), 7.28-7.20 (m, 3H), 6.45-6.30 (m, 1H), 5.33 (dt, J=10.2, 1.3 Hz, 1H), 5.05 (dt, J=17.1, 1.3 Hz, 1H), 4.96 (d, J=6.9 Hz, 1H). ¹³C NMR (101 MHz, CDCl₃) δ 152.3, 146.1, 141.4, 139.6, 136.1, 134.7, 129.3, 129.2, 128.8, 128.8, 128.1, 127.8, 127.0, 126.8, 117.7, 52.7. HRMS-ESI: m/z calculated for C₁₈H₁₆N⁺ (M+H)⁺ 246.1277, found: 246.1279. HPLC retention times (OJ-H, eluent: hexanes/i-PrOH=95/5, 254 nm, flow rate: 0.5 mL/min, T=25° C.): t_{R}=25.7 min (major), t_{R}=27.1 min (minor).

### Example 9

A method for preparing (R)-3-fluoro-5-(1-phenylallyl)pyridine, including the following steps:
S1, in a glove box filled with nitrogen, 6.5 mg of the catalyst (LA1) (0.01 mmol, 5.0 mol %), 0.5 mL of tetrahydrofuran, 38.4 mg of pinacol borane (0.30 mmol, 1.5 equiv), and 25.8 mg of 3-fluoropyridine (1j) (0.2 mmol, 1.0 equiv) were added to an 8 mL small reaction flask, stirred, and reacted at 60° C. for 5 hours to obtain 3-fluoro-1-(4,4,5,5-tetramethyl-1,3,2-dioxaborolane)-1,4-dihydropyridine, the reaction equation being as follows:

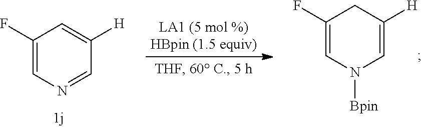

in which the structural formula of the catalyst LA1 was as follows:

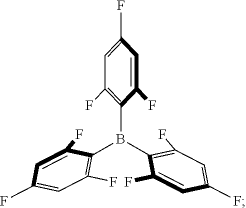

S2, iridium-catalyzed asymmetric allylation reaction of dihydropyridine: in a glove box filled with nitrogen, 3.4 mg of [Ir(COD)Cl]₂ (0.005 mmol, 2.5 mol %), 6.3 mg of the ligand L1 (0.011 mmol, 5.5 mol %), and 0.5 mL of the solvent tetrahydrofuran were added to a reaction flask and fully stirred; 22.4 mg of 1,4-diazabicyclo[2.2.2]octane (0.2 mmol, 1.0 equiv) was then added and stirred; 130.8 mg of 1-phenylallyl pivalate (0.6 mmol, 3.0 equiv) was then added; and finally, a 1,4-dihydropyridine solution cooled to room temperature was added and stirred at 0° C. for 24 hours to obtain (R)-3-fluoro-5-(1-phenylallyl)-1-(4,4,5,5-tetramethyl-1,3,2-dioxaborolane)-1,4-dihydropyridine, which was substituted with allyl in the meta-position, the reaction equation being as follows:

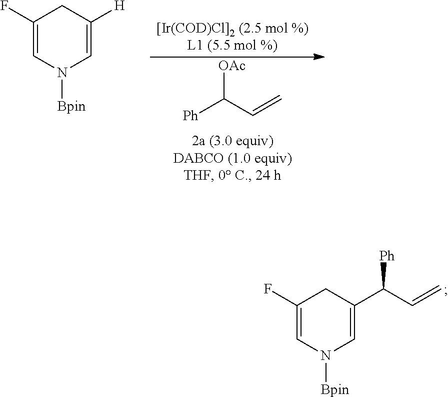

in which the structural formula of the ligand L1 was as follows:

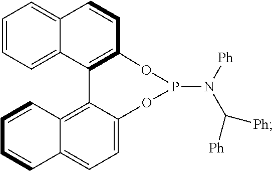

and
S3, oxidative aromatization to obtain a meta-allyl-substituted pyridine compound: the system was cooled to room temperature and stirred in air for 6 hours to generate (R)-3-fluoro-5-(1-phenylallyl)pyridine (3j), the reaction equation being as follows:

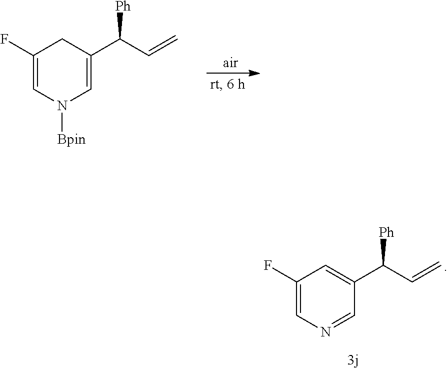

After the reaction was complete, the solvent was removed by rotary evaporation, and the residue was separated and purified by silica gel column chromatography to obtain the product (R)-3-fluoro-5-(1-phenylallyl)pyridine as a colorless liquid, with a yield of 40% and an ee value of 96%.

The product was characterized as follows:
[α]²⁰_{D}=+16.5 (c=0.4, CHCl₃). ¹H NMR (400 MHz, CDCl₃) δ 8.35-8.29 (m, 2H), 7.37-7.30 (m, 2H), 7.29-7.22 (m, 1H), 7.18-7.16 (m, 3H), 6.31-6.20 (m, 1H), 5.31 (d, J=10.2 Hz, 1H), 5.02 (d, J=17.1 Hz, 1H), 4.79 (d, J=6.9 Hz, 1H). ¹³C NMR (101 MHz, CDCl₃) δ 159.7 (d, J=252.5 Hz), 146.7, 141.4, 140.7, 139.0, 136.2 (d, J=30.3 Hz), 129.0, 128.6, 127.2, 122.9 (d, J=20.3 Hz), 117.9, 52.1. ¹⁹F NMR (376 MHz, CDCl₃) δ-127.0. HRMS-ESI: m/z calculated for C₁₄H₁₃FN⁺ (M+H)⁺ 214.1027, found: 214.1027. HPLC retention times (AD-H, eluent: hexanes/i-PrOH=95/5, 254 nm, flow rate: 0.5 mL/min, T=25° C.): t_{R}=16.0 min (major), t_{R}=17.0 min (minor).

### Example 10

A method for preparing ethyl 4-(8-chloro-3-(1-phenylallyl)-5,6-dihydro-11H-benzo[5,6]cyclohepta[1,2-b]pyridin-11-ylidene)-1-piperidinecarboxylate, including the following steps:
S1, in a glove box filled with nitrogen, 6.5 mg of the catalyst (LA2) (0.01 mmol, 5.0 mol %), 1 mL of trichloromethane, 30.7 mg of pinacol borane (0.24 mmol, 1.2 equiv), and 76.6 mg of loratadine (1k) (0.2 mmol, 1.0 equiv) were added to an 8 mL small reaction flask, stirred, and reacted at 60° C. for 12 hours to obtain ethyl 4-(8-chloro-1-(4,4,5,5-tetramethyl-1,3,2-dioxaborolane)-1,4,5,6-tetrahydro-11H-benzo[5,6]cyclo hepta[1,2-b]pyridin-11-ylidene)-1-piperidinecarboxylate, the reaction equation being as follows:

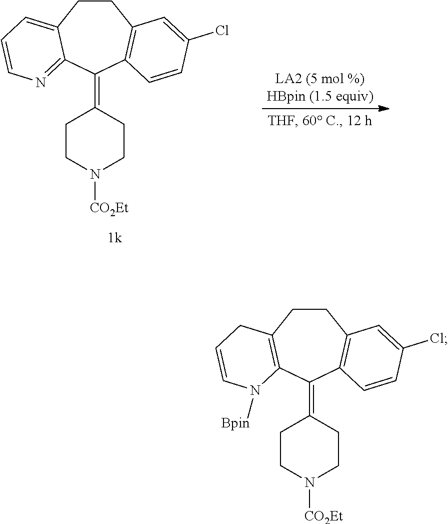

in which the structural formula of the catalyst LA2 was as follows:

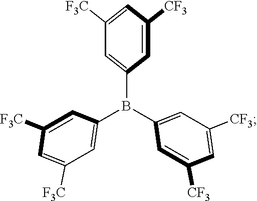

S2, iridium-catalyzed asymmetric allylation reaction of dihydropyridine: in a glove box filled with nitrogen, 3.4 mg of [Ir(COD)Cl]₂ (0.005 mmol, 2.5 mol %), 6.3 mg of the ligand L1 (0.011 mmol, 5.5 mol %), and 0.5 mL of the solvent tetrahydrofuran were added to a reaction flask and fully stirred; 22.4 mg of 1,4-diazabicyclo[2.2.2]octane (0.2 mmol, 1.0 equiv) was then added and stirred; 130.8 mg of 1-phenylallyl pivalate (0.6 mmol, 3.0 equiv) was then added; and finally, a 1,4-dihydropyridine solution cooled to room temperature was added and stirred at 0° C. for 24 hours to obtain ethyl (R)-4-(8-chloro-3-(1-phenylallyl)-1-(4,4,5,5-tetramethyl-1,3,2-dioxaborolane)-1,4,5,6-tetrahydro-11H-benzo[5,6]cyclohepta[1,2-b]pyridin-11-ylidene)-1-piperidinecarboxylate, which was substituted with allyl in the meta-position, the reaction equation being as follows:

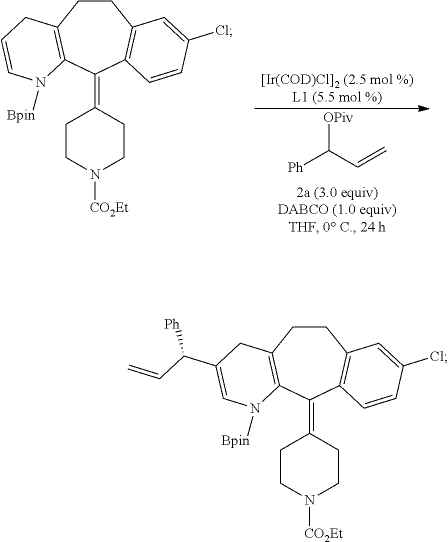

S3, oxidative aromatization to obtain a meta-allyl-substituted pyridine compound: the system was cooled to room temperature and stirred in air for 6 hours to generate ethyl (R)-4-(8-chloro-3-(1-phenylallyl)-5,6-dihydro-11H-benzo[5,6]cyclohepta-[1,2-b]pyridin-11-ylide ne)piperidine-1-carboxylate (3k), the reaction equation being as follows:

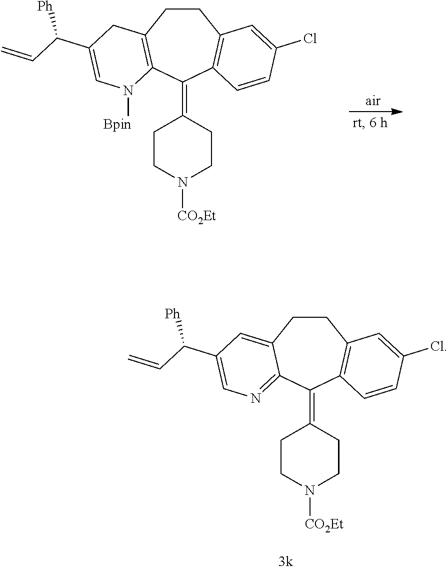

After the reaction was complete, the solvent was removed by rotary evaporation, and the residue was separated and purified by silica gel column chromatography to obtain the product ethyl
(R)-4-(8-chloro-3-(1-phenylallyl)-1,4,5,6-tetrahydro-11H-benzo[5,6]cyclohepta[1,2-b]pyridin-11-ylidene)-1-piperidinecarboxylate as a white solid, with a yield of 38% and an ee value of 97%.

The product was characterized as follows:
[α]²⁰_{D}=+6.8 (c=0.80, CHCl₃). ¹H NMR (400 MHz, CDCl₃) δ 8.26 (d, J=2.1 Hz, 1H), 7.35-7.26 (m, 2H), 7.26-7.03 (m, 7H), 6.30-6.18 (m, 1H), 5.30-5.20 (m, 1H), 4.90-5.03 (m, 1H), 4.68 (d, J=7.1 Hz, 1H), 4.13 (q, J=7.1 Hz, 2H), 3.70-3.90 (m, 2H), 3.40-3.20 (m, 2H), 3.19-3.10 (m, 2H), 2.85-2.70 (m, 2H), 2.57-2.42 (m, 1H), 2.40-2.24 (m, 3H), 1.25 (t, J=7.1 Hz, 3H). ¹³C NMR (101 MHz, CDCl₃) δ 155.6, 155.0, 146.9, 142.2, 142.1, 139.8, 139.7, 139.6, 138.2, 137.7, 137.7, 137.3, 134.1, 133.0, 130.5, 129.0, 128.8, 128.6, 126.9, 126.3, 117.3, 61.5, 52.3, 44.9, 31.8, 31.8, 31.7, 30.9, 30.7, 14.8. HRMS-ESI: m/z calculated for C₃₁H₃₂ClN₂O₂⁺ (M+H)⁺ 499.2147, found: 499.2146. HPLC retention times (AD-H, eluent: hexanes/i-PrOH=90/10, 254 nm, flow rate: 0.5 mL/min, T=25° C.): t_{R}=24.6 min (major), t_{R}=38.6 min (minor).

### Example 11

A method for preparing 2-((2-(4-chlorophenoxy)-2-methylpropyl)oxy)ethyl (R)-5-(1-phenylallyl)pyridine-3-carboxylate, including the following steps:
S1, in a glove box filled with nitrogen, 4.0 mg of the catalyst (LA1) (0.01 mmol, 5.0 mol %), 1 mL of toluene, 38.4 mg of pinacol borane (0.30 mmol, 1.5 equiv), and 72.8 mg of etofibrate (11) (0.2 mmol, 1.0 equiv) were added to an 8 mL small reaction flask, stirred, and reacted at 80° C. for 12 hours to obtain 2-((2-(4-chlorophenoxy)-2-methylpropanoyl)oxy)ethyl 1-(4,4,5,5-tetramethyl-1,3,2-dioxaborolane)-1,4-dihydropyridine-3-carboxylate, the reaction equation being as follows:

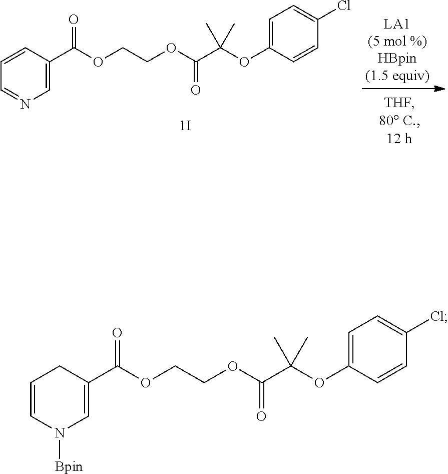

in which the structural formula of the catalyst LA1 was as follows:

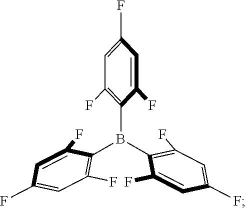

S2, iridium-catalyzed asymmetric allylation reaction of dihydropyridine: in a glove box filled with nitrogen, 3.4 mg of [Ir(COD)Cl]₂ (0.005 mmol, 2.5 mol %), 6.3 mg of the ligand L1 (0.011 mmol, 5.5 mol %), and 0.5 mL of the solvent tetrahydrofuran were added to a reaction flask and fully stirred; 22.4 mg of 1,4-diazabicyclo[2.2.2]octane (0.2 mmol, 1.0 equiv) was then added and stirred; 87.2 mg of 1-phenylallyl pivalate (0.4 mmol, 2.0 equiv) was then added; and finally, a 1,4-dihydropyridine solution cooled to room temperature was added and stirred at 0° C. for 24 hours to obtain 2-((2-(4-chlorophenoxy)-2-methylpropyl)oxy)ethyl (R)-1-(4,4,5,5-tetramethyl-1,3,2-dioxaborolane)-5-(1-phenylallyl)-1,4-dihydropyridine-3-carbox ylate, which was substituted with allyl in the meta-position, the reaction equation being as follows:

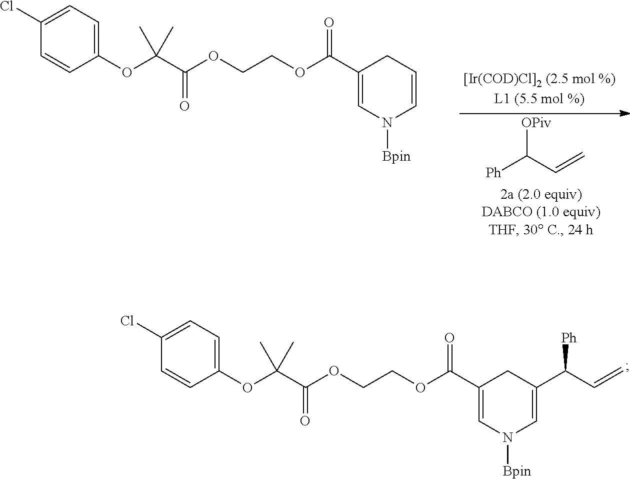

S3, oxidative aromatization to obtain a meta-allyl-substituted pyridine compound: the system was cooled to room temperature and stirred in air for 6 hours to generate 2-((2-(4-chlorophenoxy)-2-methylpropanoyl)oxy)ethyl (R)-5-(1-phenylallyl)nicotinate (3l), the reaction equation being as follows:

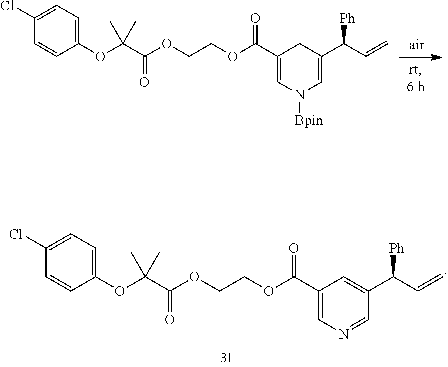

After the reaction was complete, the solvent was removed by rotary evaporation, and the residue was separated and purified by silica gel column chromatography to obtain the product 2-((2-(4-chlorophenoxy)-2-methylpropyl)oxy)ethyl (R)-5-(1-phenylallyl)pyridine-3-carboxylate as a colorless liquid, with a yield of 61% and an ee value of 99%.

The product was characterized as follows:
[α]²⁰_{D}=−9.3 (c=0.20, CHCl₃). ¹H NMR (400 MHz, CDCl₃) δ 8.92 (s, 1H), 8.56 (s, 1H), 7.97 (s, 1H), 7.28-7.22 (m, 2H), 7.20-7.15 (m, 1H), 7.12-7.05 (m, 2H), 7.01 (d, J=8.9 Hz, 2H), 6.67 (d, J=8.9 Hz, 2H), 6.30-6.15 (m, 1H), 5.24 (d, J=10.2 Hz, 1H), 4.93 (d, J=17.0 Hz, 1H), 4.73 (d, J=7.0 Hz, 1H), 4.48-4.42 (m, 4H), 1.49 (s, 6H). ¹³C NMR (151 MHz, CDCl₃) δ 174.0, 165.1, 154.3, 154.0, 149.1, 141.4, 139.0, 138.9, 137.0, 129.3, 129.0, 128.6, 127.5, 127.2, 125.4, 120.5, 118.0, 79.5, 63.0, 52.4, 29.8, 25.4. HRMS-ESI: m/z calculated for C₂₇H₂₇ClNO₅⁺ (M+H)⁺ 480.1572, found: 480.1578. HPLC retention times (AD-H, eluent: hexanes/i-PrOH=90/10, 254 nm, flow rate: 0.5 mL/min, T=25° C.): t_{R}=25.4 min (major), t_{R}=27.7 min (minor).

### Example 12

A method for preparing (R)-3-phenyl-5-(1-p-tolylallyl)pyridine, including the following steps:
S1, hydroboration reaction to prepare dihydropyridine: in a glove box filled with nitrogen, the catalyst B(2,4,6-F₃C₆H₂)₃ (LA1) (4.0 mg, 0.01 mmol, 5.0 mol %), 0.5 mL of tetrahydrofuran, 38.4 mg of pinacol borane (0.30 mmol, 1.5 equiv), and 31.0 mg of 3-phenylpyridine (1a) (0.2 mmol, 1.0 equiv) were added to an 8 mL small reaction flask, stirred, and reacted at 80° C. for 12 hours to obtain 3-phenyl-1-(4,4,5,5-tetramethyl-1,3,2-dioxaborolane)-1,4-dihydropyridine, the reaction equation being as follows:

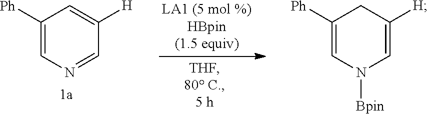

in which the structural formula of the catalyst LA1 was as follows:

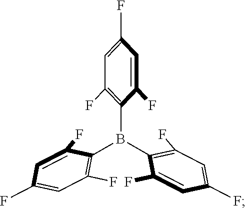

S2, iridium-catalyzed asymmetric allylation reaction of dihydropyridine: in a glove box filled with nitrogen, 3.4 mg of [Ir(COD)Cl]₂ (0.005 mmol, 2.5 mol %), 6.3 mg of the ligand L1 (0.011 mmol, 5.5 mol %), and 0.5 mL of the solvent tetrahydrofuran were added to a reaction flask and fully stirred; 22.4 mg of 1,4-diazabicyclo[2.2.2]octane (0.2 mmol, 1.0 equiv) was then added and stirred; 92.8 mg of 1-p-tolylallyl pivalate (0.4 mmol, 2.0 equiv) was then added; and finally, a 1,4-dihydropyridine solution cooled to room temperature was added and stirred at 30° C. for 16 hours to obtain (R)-1-(4,4,5,5-tetramethyl-1,3,2-dioxaborolane)-3-phenyl-5-(1-p-tolylallyl)-1,4-dihydropyridine, which was substituted with allyl in the meta-position, the reaction equation being as follows:

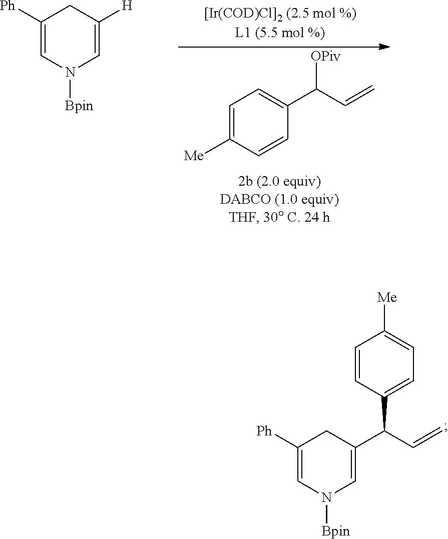

in which the structural formula of the ligand L1 was as follows:

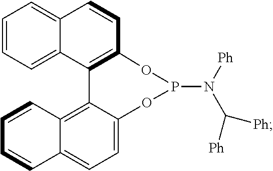

and
S3, oxidative aromatization to obtain a meta-allyl-substituted pyridine compound: the system was cooled to room temperature and stirred in air for 1 hour to generate (R)-3-phenyl-5-(1-(p-tolyl)allyl)pyridine (4b), the reaction equation being as follows:

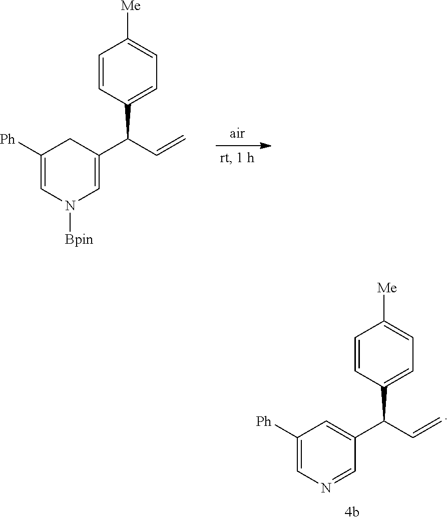

After the reaction was complete, the solvent was removed by rotary evaporation, and the residue was separated and purified by silica gel column chromatography to obtain the product (R)-3-phenyl-5-(1-p-tolylallyl)pyridine as a colorless liquid, with a yield of 71% and an ee value of 95%.

The product was characterized as follows:
[α]²⁰_{D}=−14.7 (c=0.77, CHCl₃). ¹H NMR (400 MHz, CDCl₃) δ 8.70 (d, J=2.1 Hz, 1H), 8.45 (d, J=2.1 Hz, 1H), 7.66 (t, J=2.1 Hz, 1H), 7.56-7.51 (m, 2H), 7.48-7.41 (m, 2H), 7.42-7.35 (m, 1H), 7.18-7.06 (m, 4H), 6.36-6.25 (m, 1H), 5.29 (d, J=10.2 Hz, 1H), 5.04 (d, J=17.1 Hz, 1H), 4.79 (d, J=7.0 Hz, 1H), 2.33 (s, 3H). ¹³C NMR (101 MHz, CDCl₃) δ 148.9, 146.4, 139.8, 139.1, 138.9, 138.0, 136.6, 136.5, 134.6, 129.5, 129.1, 128.5, 128.2, 127.4, 117.3, 52.2, 21.1. HRMS-ESI: m/z calculated for C₂₁H₂₀N⁺ (M+H)⁺ 286.1590, found: 286.1584. HPLC retention times (OJ-H, eluent: hexanes/i-PrOH=90/10, 254 nm, flow rate: 0.5 mL/min, T=25° C.): t_{R}=13.7 min (major), t_{R}=18.6 min (minor).

### Example 13

A method for preparing (R,E)-3-phenyl-5-(1-phenylpenta-1,4-dien-3-yl)pyridine, including the following steps:
S1, hydroboration reaction to prepare dihydropyridine: in a glove box filled with nitrogen, the catalyst B(2,4,6-F₃C₆H₂)₃ (LA1) (4.0 mg, 0.01 mmol, 5.0 mol %), 0.5 mL of tetrahydrofuran, 38.4 mg of pinacol borane (0.30 mmol, 1.5 equiv), and 31.0 mg of 3-phenylpyridine (1a) (0.2 mmol, 1.0 equiv) were added to an 8 mL small reaction flask, stirred, and reacted at 80° C. for 12 hours to obtain 3-phenyl-1-(4,4,5,5-tetramethyl-1,3,2-dioxaborolane)-1,4-dihydropyridine, the reaction equation being as follows:

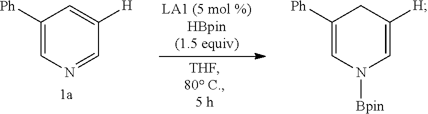

in which the structural formula of the catalyst LA1 was as follows:

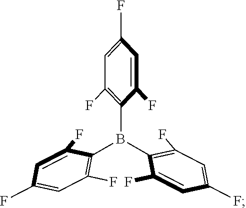

S2, iridium-catalyzed asymmetric allylation reaction of dihydropyridine: in a glove box filled with nitrogen, 3.4 mg of [Ir(COD)Cl]₂ (0.005 mmol, 2.5 mol %), 6.3 mg of the ligand L1 (0.011 mmol, 5.5 mol %), and 0.5 mL of the solvent tetrahydrofuran were added to a reaction flask and fully stirred; 22.4 mg of 1,4-diazabicyclo[2.2.2]octane (0.2 mmol, 1.0 equiv) was then added and stirred; 146.4 mg of (E)-1-phenylpenta-1,4-dien-3-yl pivalate (0.6 mmol, 3.0 equiv) was then added; and finally, a 1,4-dihydropyridine solution cooled to room temperature was added and stirred at 5° C. for 24 hours to obtain (R,E)-3-phenyl-5-(1-phenylpenta-1,4-dien-3-yl)-1-(4,4,5,5-tetramethyl-1,3,2-dioxaborolane)-1,4-dihydropyridine, which was substituted with allyl in the meta-position, the reaction equation being as follow

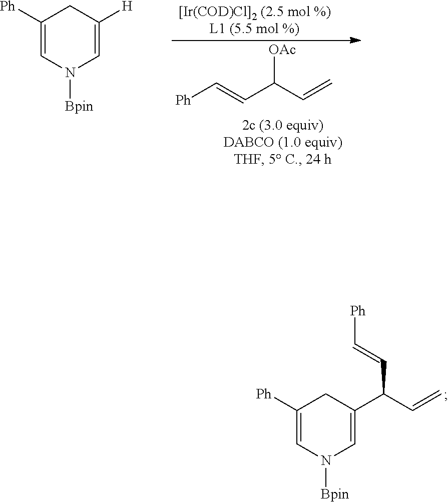

in which the structural formula of the ligand L1 was as follows:

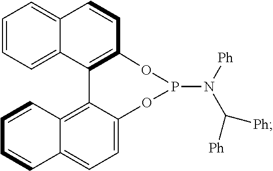

and
S3, oxidative aromatization to obtain a meta-allyl-substituted pyridine compound:
the system was cooled to room temperature and stirred in air for 1 hour to generate (R,E)-3-phenyl-5-(1-phenylpenta-1,4-dien-3-yl)pyridine (4c), the reaction equation being as follows:

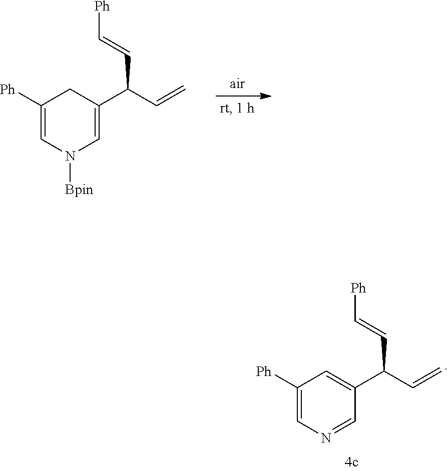

After the reaction was complete, the solvent was removed by rotary evaporation, and the residue was separated and purified by silica gel column chromatography to obtain the product (R,E)-3-phenyl-5-(1-phenylpenta-1,4-dien-3-yl)pyridine as a colorless liquid, with a yield of 52% and an ee value of 96%.

The product was characterized as follows:
[α]²⁰_{D}=−2.4 (c=0.44, CHCl₃). ¹H NMR (400 MHz, CDCl₃) δ 8.73 (d, J=2.0 Hz, 1H), 8.52 (d, J=2.0 Hz, 1H), 7.75 (t, J=2.0 Hz, 1H), 7.62-7.55 (m, 2H), 7.50-7.44 (m, 2H), 7.42-7.35 (m, 3H), 7.34-7.26 (m, 2H), 7.26-7.21 (m, 1H), 6.53-6.35 (m, 2H), 6.20-6.08 (m, 1H), 5.28 (dt, J=10.2, 1.3 Hz, 1H), 5.21 (dt, J=17.0, 1.3 Hz, 1H), 4.36-4.30 (m, 1H). ¹³C NMR (101 MHz, CDCl₃) δ 148.5, 146.7, 139.0, 138.1, 137.9, 137.0, 136.7, 134.2, 131.8, 130.5, 129.2, 128.7, 128.3, 127.7, 127.4, 126.5, 117.0, 49.9. HRMS-ESI: m/z calculated for C₂₂H₂₀N⁺ (M+H)⁺ 298.1590. found: 298.1594. HPLC retention times (OJ-H, eluent: hexanes/i-PrOH=95/5, 280 nm, flow rate: 0.5 mL/min, T=25° C.): t_{R}=33.5 min (major), t_{R}=44.5 min (minor).

### Example 14

A method for preparing (S)-3-(1-octen-3-yl)-5-phenylpyridine, including the following steps:
S1, hydroboration reaction to prepare dihydropyridine: in a glove box filled with nitrogen, the catalyst B(2,4,6-F₃C₆H₂)₃ (LA1) (4.0 mg, 0.01 mmol, 5.0 mol %), 0.3 mL of tetrahydrofuran, 38.4 mg of pinacol borane (0.30 mmol, 1.5 equiv), and 31.0 mg of 3-phenylpyridine (1a) (0.2 mmol, 1.0 equiv) were added to an 8 mL small reaction flask, stirred, and reacted at 80° C. for 12 hours to obtain 3-phenyl-1-(4,4,5,5-tetramethyl-1,3,2-dioxaborolane)-1,4-dihydropyridine, the reaction equation being as follows:

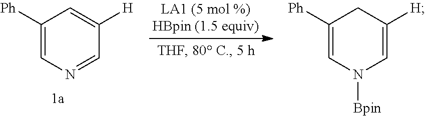

in which the structural formula of the catalyst LA1 was as follows:

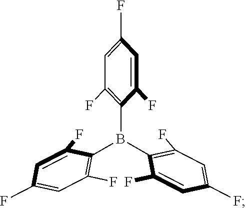

S2, iridium-catalyzed asymmetric allylation reaction of dihydropyridine: in a glove box filled with nitrogen, 3.4 mg of [Ir(COD)Cl]₂ (0.005 mmol, 2.5 mol %), 6.3 mg of the ligand L1 (0.011 mmol, 5.5 mol %), and 0.3 mL of the solvent tetrahydrofuran were added to a reaction flask and fully stirred; 22.4 mg of 1,4-diazabicyclo[2.2.2]octane (0.02 mmol, 0.1 equiv) was then added and stirred; 74.4 mg of methyl 1-octen-3--ylcarbonate (0.4 mmol, 2.0 equiv) was then added; and finally, a 1,4-dihydropyridine solution cooled to room temperature was added and stirred at 50° C. for 24 hours to obtain (S)-3-(1-octen-3-yl)-5-phenyl-1-(4,4,5,5-tetramethyl-1,3,2-dioxaborolane)-1,4-dihydropyridine, which was substituted with allyl in the meta-position, the reaction equation being as follows:

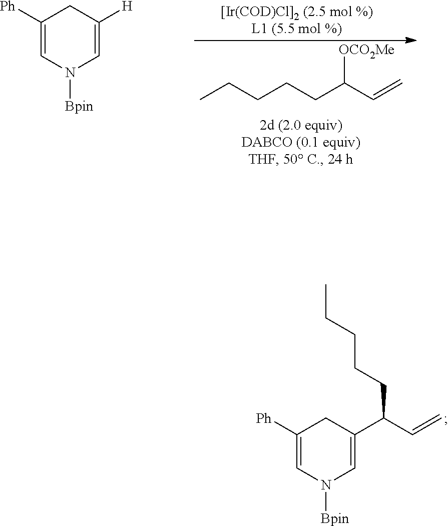

in which the structural formula of the ligand L1 was as follows:

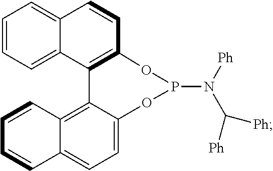

and
S3, oxidative aromatization to obtain a meta-allyl-substituted pyridine compound: the system was cooled to room temperature and stirred in air for 1 hour to generate (S)-3-(oct-1-en-3-yl)-5-phenylpyridine (4d), the reaction equation being as follows:

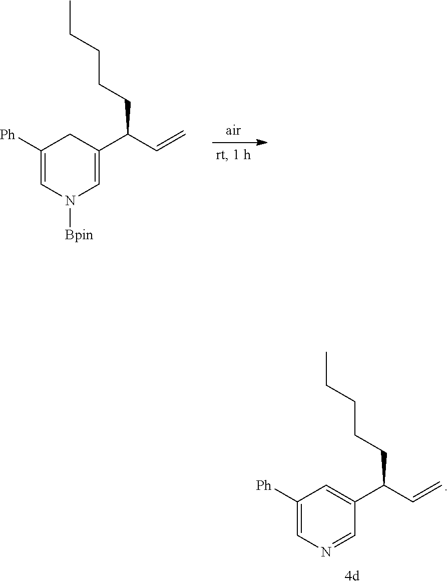

After the reaction was complete, the solvent was removed by rotary evaporation, and the residue was separated and purified by silica gel column chromatography to obtain the product (S)-3-(1-octen-3-yl)-5-phenylpyridine as a colorless liquid, with a yield of 52% and an ee value of 96%.

The product was characterized as follows:
[α]²⁰_{D}=−2.4 (c=0.44, CHCl₃). ¹H NMR (400 MHz, CDCl₃) δ 8.73 (d, J=2.0 Hz, 1H), 8.52 (d, J=2.0 Hz, 1H), 7.75 (t, J=2.0 Hz, 1H), 7.62-7.55 (m, 2H), 7.50-7.44 (m, 2H), 7.42-7.35 (m, 3H), 7.34-7.26 (m, 2H), 7.26-7.21 (m, 1H), 6.53-6.35 (m, 2H), 6.20-6.08 (m, 1H), 5.28 (dt, J=10.2, 1.3 Hz, 1H), 5.21 (dt, J=17.0, 1.3 Hz, 1H), 4.36-4.30 (m, 1H). ¹³C NMR (101 MHz, CDCl₃) δ 148.5, 146.7, 139.0, 138.1, 137.9, 137.0, 136.7, 134.2, 131.8, 130.5, 129.2, 128.7, 128.3, 127.7, 127.4, 126.5, 117.0, 49.9. HRMS-ESI: m/z calculated for C₂₂H₂₀N⁺ (M+H)⁺ 298.1590. found: 298.1594. HPLC retention times (OJ-H, eluent: hexanes/i-PrOH=95/5, 280 nm, flow rate: 0.5 mL/min, T=25° C.): t_{R}=33.5 min (major), t_{R}=44.5 min (minor).

### Example 15

A method for preparing (S,E)-3-phenyl-5-(4-phenyl-3-buten-2-yl)pyridine, including the following steps:
S1, hydroboration reaction to prepare dihydropyridine: in a glove box filled with nitrogen, the catalyst B(2,4,6-F₃C₆H₂)₃ (LA1) (8.0 mg, 0.02 mmol, 10.0 mol %), 1 mL of tetrahydrofuran, 38.4 mg of pinacol borane (0.30 mmol, 1.5 equiv), and 31.0 mg of 3-phenylpyridine (1a) (0.2 mmol, 1.0 equiv) were added to an 8 mL small reaction flask, stirred, and reacted at 80° C. for 12 hours to obtain 3-phenyl-1-(4,4,5,5-tetramethyl-1,3,2-dioxaborolane)-1,4-dihydropyridine, the reaction equation being as follows:

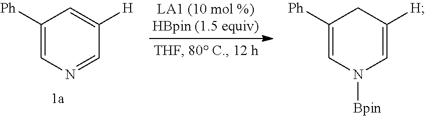

in which the structural formula of the catalyst LA1 was as follows:

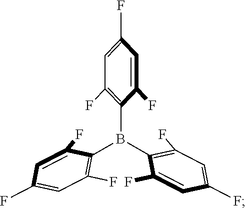

S2, palladium-catalyzed asymmetric allylation reaction of dihydropyridine: in a glove box filled with nitrogen, 5.1 mg of Pd(P^{t}Bu₃)₂ (0.01 mmol, 5 mol %), 6.8 mg of the ligand L4 (0.011 mmol, 5.5 mol %), and 1 mL of the solvent tetrahydrofuran were added to a reaction flask and fully stirred; subsequently, 57 mg of (E)-4-phenyl-3-buten-2-yl carboxylate (0.3 mmol, 1.5 equiv), the well-stirred palladium catalyst solution, and 44.8 mg of 1,4-diazabicyclo[2.2.2]octane (0.3 mmol, 1.5 equiv) were successively added to a 1,4-dihydropyridine solution cooled to room temperature and stirred in a nitrogen atmosphere at 60° C. for 24 hours to obtain (S,E)-3-phenyl-5-(4-phenyl-3-buten-2-yl)-1-(4,4,5,5-tetramethyl-1,3,2-dioxaborolane)-1,4-dihyd ropyridine, which was substituted with allyl in the meta-position, the reaction equation being as follows:

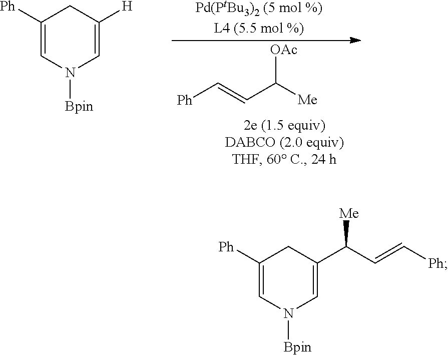

in which the structural formula of the ligand L4 was as follows:

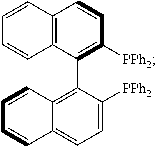

and
S3, oxidative aromatization to obtain a meta-allyl-substituted pyridine compound:
the system was cooled to room temperature and stirred in air for 1 hour to generate (S,E)-3-phenyl-5-(4-phenylbut-3-en-2-yl)pyridine (4e), the reaction equation being as follows:

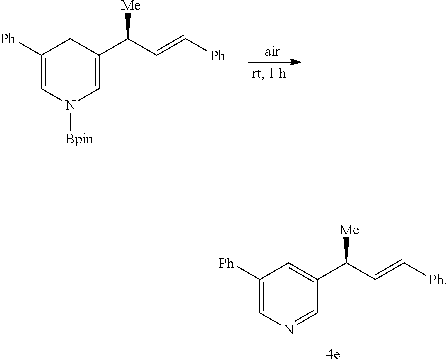

After the reaction was complete, the solvent was removed by rotary evaporation, and the residue was separated and purified by silica gel column chromatography to obtain the product (S,E)-3-phenyl-5-(4-phenyl-3-buten-2-yl)pyridine as a colorless liquid, with a yield of 77% and an ee value of 93%.

The product was characterized as follows:
[α]²⁵D=+7.0 (c=0.75, CHCl₃). ¹H NMR (400 MHz, CDCl₃) δ 8.73 (d, J=2.0 Hz, 1H), 8.54 (d, J=1.8 Hz, 1H), 7.75 (t, J=2.1 Hz, 1H), 7.64-7.56 (m, 2H), 7.52-7.45 (m, 2H), 7.44-7.35 (m, 3H), 7.31 (t, J=7.5 Hz, 2H), 7.25-7.20 (m, 1H), 6.49 (d, J=15.9 Hz, 1H), 6.40 (dd, J=15.9, 6.4 Hz, 1H), 3.81-3.72 (m, 1H), 1.56 (d, J=7.0 Hz, 3H). ¹³C NMR (101 MHz, CDCl₃) δ 148.1, 146.5, 140.8, 138.1, 137.2, 136.6, 133.9, 133.3, 129.7, 129.1, 128.7, 128.2, 127.5, 127.4, 126.3, 40.3, 21.2. HRMS (ESI) calcd for C₂₁H₂₀N⁺ (M+H)⁺: 286.1590, found: 286.1590. HPLC retention times (OJ-H, eluent: Hexanes/i-PrOH=90/10, 254 nm, flow rate: 0.5 mL/min, T=25° C.): t_{R}=23.8 min (minor), t_{R}=26.6 min (major).

### Example 16

A method for preparing (S,E)-3-(4-(3-furyl)-3-buten-2-yl)-5-phenylpyridine, including the following steps:
S1, hydroboration reaction to prepare dihydropyridine: in a glove box filled with nitrogen, the catalyst B(2,4,6-F₃C₆H₂)₃ (LA1) (8.0 mg, 0.02 mmol, 10.0 mol %), 1 mL of tetrahydrofuran, 38.4 mg of pinacol borane (0.30 mmol, 1.5 equiv), and 31.0 mg of 3-phenylpyridine (1a) (0.2 mmol, 1.0 equiv) were added to an 8 mL small reaction flask, stirred, and reacted at 80° C. for 12 hours to obtain 3-phenyl-1-(4,4,5,5-tetramethyl-1,3,2-dioxaborolane)-1,4-dihydropyridine, the reaction equation being as follows:

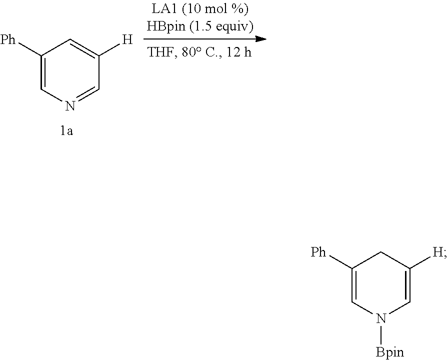

in which the structural formula of the catalyst LA1 was as follows:

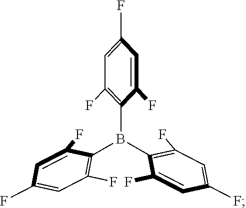

S2, palladium-catalyzed asymmetric allylation reaction of dihydropyridine: 5.1 mg of Pd(P^{t}Bu₃)₂ (0.01 mmol, 5 mol %), 6.8 mg of the ligand L4 (0.011 mmol, 5.5 mol %), and 1 mL of the solvent tetrahydrofuran were added to a reaction flask and fully stirred; subsequently, 54 mg of (E)-4-(3-furyl)-3-buten-2-yl carboxylate (0.3 mmol, 1.5 equiv), the well-stirred palladium catalyst solution, and 44.8 mg of 1,4-diazabicyclo[2.2.2]octane (0.3 mmol, 1.5 equiv) were successively added to a 1,4-dihydropyridine solution cooled to room temperature and stirred in a nitrogen atmosphere at 50° C. for 24 hours to obtain (S,E)-3-(4-(3-furyl)-3-buten-2-yl)-5-phenyl-1-(4,4,5,5-tetramethyl-1,3,2-dioxaborolane)-1,4-dihy dropyridine, which was substituted with allyl in the meta-position, the reaction equation being as follows:

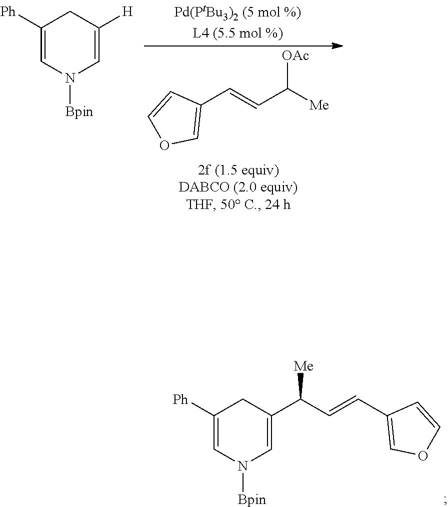

in which the structural formula of the ligand L4 was as follows:

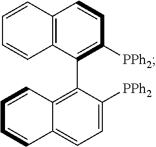

and
S3, oxidative aromatization to obtain a meta-allyl-substituted pyridine compound:
the system was cooled to room temperature and stirred in air for 1 hour to generate (S,E)-3-(4-(furan-3-yl)but-3-en-2-yl)-5-phenylpyridine (4f), the reaction equation being as follows:

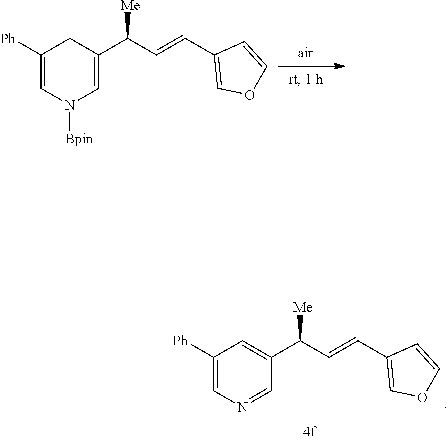

After the reaction was complete, the solvent was removed by rotary evaporation, and the residue was separated and purified by silica gel column chromatography to obtain the product (S,E)-3-(4-(3-furyl)-3-buten-2-yl)-5-phenylpyridine as a colorless liquid, with a yield of 67% and an ee value of 90%.

The product was characterized as follows:
[α]²⁶_{D}=+20.7 (c=0.55, CHCl₃). ¹H NMR (400 MHz, CDCl₃) δ 8.71 (d, J=2.1 Hz, 1H), 8.50 (d, J=2.0 Hz, 1H), 7.72 (t, J=2.1 Hz, 1H), 7.62-7.55 (m, 2H), 7.50-7.44 (m, 2H), 7.43-7.37 (m, 2H), 7.35 (s, 1H), 6.50 (d, J=1.7 Hz, 1H), 6.31 (d, J=16.0 Hz, 1H), 6.11 (dd, J=16.0, 6.6 Hz, 1H), 3.76-3.62 (m, 1H), 1.51 (d, J=7.1 Hz, 3H). ¹³C NMR (101 MHz, CDCl₃) δ 148.1, 146.5, 143.7, 140.9, 140.3, 138.1, 136.6, 133.5, 133.3, 129.2, 128.2, 127.4, 124.1, 119.4, 107.6, 40.2, 21.1. HRMS (ESI) calcd. for C₁₉H₁₈NO⁺ (M+H)⁺: 276.1383, found: 276.1385. HPLC retention times (IF, eluent: Hexanes/i-PrOH=90/10, 254 nm, flow rate: 0.5 mL/min, T=25° C.): t_{R}=34.2 min (minor), t_{R}=38.7 min (major).

### Example 17

A method for preparing 3-phenyl-5-((S,3E,5E)-6-phenyl-3,5-hexadien-2-yl)pyridine, including the following steps:
S1, hydroboration reaction to prepare dihydropyridine: in a glove box filled with nitrogen, the catalyst B(2,4,6-F₃C₆H₂)₃ (LA1) (8.0 mg, 0.02 mmol, 10.0 mol %), 1 mL of tetrahydrofuran, 38.4 mg of pinacol borane (0.30 mmol, 1.5 equiv), and 31.0 mg of 3-phenylpyridine (1a) (0.2 mmol, 1.0 equiv) were added to an 8 mL small reaction flask, stirred, and reacted at 80° C. for 12 hours to obtain 3-phenyl-1-(4,4,5,5-tetramethyl-1,3,2-dioxaborolane)-1,4-dihydropyridine, the reaction equation being as follows:

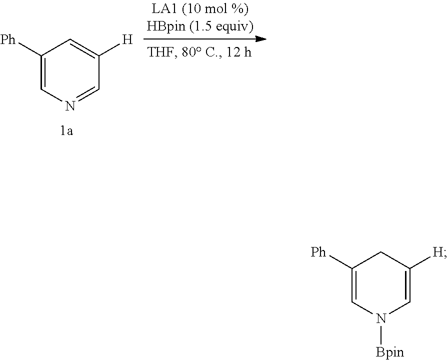

in which the structural formula of the catalyst LA1 was as follows:

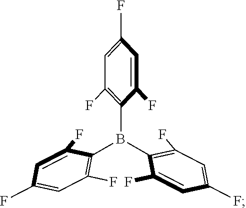

S2, palladium-catalyzed asymmetric allylation reaction of dihydropyridine: 5.1 mg of Pd(P^{t}Bu₃)₂ (0.01 mmol, 5 mol %), 6.8 mg of the ligand L4 (0.011 mmol, 5.5 mol %), and 1 mL of the solvent tetrahydrofuran were added to a reaction flask and fully stirred; subsequently, 65 mg of (3E,5E)-6-phenyl-3,5-hexadien-2-yl carboxylate (0.3 mmol, 1.5 equiv), the well-stirred palladium catalyst solution, and 44.8 mg of 1,4-diazabicyclo[2.2.2]octane (0.3 mmol, 1.5 equiv) were successively added to a 1,4-dihydropyridine solution cooled to room temperature and stirred in a nitrogen atmosphere at 50° C. for 24 hours to obtain 3-phenyl-5-((S,3E,5E)-6-phenyl-3,5-hexadien-2-yl)-1-(4,4,5,5-tetramethyl-1,3,2-dioxaborolane)-1,4-dihydropyridine, which was substituted with allyl in the meta-position, the reaction equation being as follows:

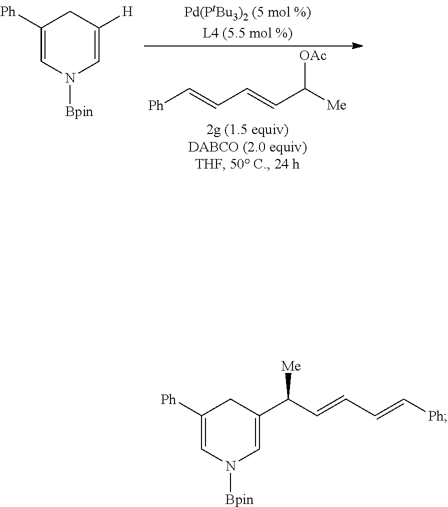

in which the structural formula of the ligand L4 was as follows:

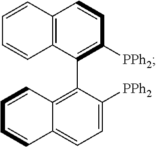

S3, oxidative aromatization to obtain a meta-allyl-substituted pyridine compound:
the system was cooled to room temperature and stirred in air for 1 hour to generate 3-phenyl-5-((S,3E,5E)-6-phenylhexa-3,5-dien-2-yl)pyridine (4g), the reaction equation being as follows:

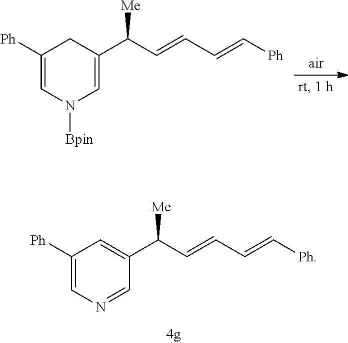

After the reaction was complete, the solvent was removed by rotary evaporation, and the residue was separated and purified by silica gel column chromatography to obtain the product 3-phenyl-5-((S,3E,5E)-6-phenyl-3,5-hexadien-2-yl)pyridine as a colorless liquid, with a yield of 60% and an ee value of 94%.

The product was characterized as follows:
[α]²⁸D=+25.2 (c=0.4, CHCl₃). ¹H NMR (400 MHz, CDCl₃) δ 8.71 (s, 1H), 8.49 (s, 1H), 7.71 (s, 1H), 7.59 (d, J=7.7 Hz, 2H), 7.48 (t, J=7.5 Hz, 2H), 7.44-7.35 (m, 3H), 7.30 (t, J=7.5 Hz, 2H), 7.21 (t, J=7.1 Hz, 1H), 6.78 (dd, J=15.6, 10.4 Hz, 1H), 6.52 (d, J=15.6 Hz, 1H), 6.28 (dd, J=15.0, 10.5 Hz, 1H), 6.00 (dd, J=15.2, 6.7 Hz, 1H), 3.77-3.60 (m, 1H), 1.50 (d, J=6.9 Hz, 3H). ¹³C NMR (101 MHz, CDCl₃) δ 148.0, 146.4, 140.9, 138.2, 138.1, 137.4, 136.6, 133.3, 131.9, 130.3, 129.2, 128.8, 128.7, 128.2, 127.6, 127.4, 126.4, 40.1, 21.2. HRMS (ESI) calcd. for C₂₃H₂₂N⁺ (M+H)⁺: 312.1747, found: 312.1748. HPLC retention times (IC-3, eluent: Hexanes/i-PrOH=95/5, 254 nm, flow rate: 0.5 mL/min, T=25° C.): t_{R}=45.5 min (minor), t_{R}=51.7 min (major).

### Example 18

A method for preparing (S,E)-3-(3-hepten-2-yl)-5-phenylpyridine, including the following steps:
S1, hydroboration reaction to prepare dihydropyridine: in a glove box filled with nitrogen, the catalyst B(2,4,6-F₃C₆H₂)₃ (LA1) (8.0 mg, 0.02 mmol, 10.0 mol %), 1 mL of toluene, 38.4 mg of pinacol borane (0.30 mmol, 1.5 equiv), and 31.0 mg of 3-phenylpyridine (1a) (0.2 mmol, 1.0 equiv) were added to an 8 mL small reaction flask, stirred, and reacted at 80° C. for 12 hours to obtain 3-phenyl-1-(4,4,5,5-tetramethyl-1,3,2-dioxaborolane)-1,4-dihydropyridine, the reaction equation being as follows:

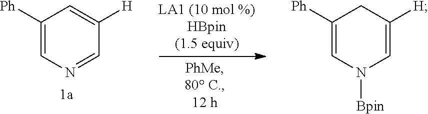

in which the structural formula of the catalyst LA1 was as follows:

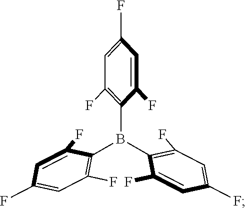

S2, palladium-catalyzed asymmetric allylation reaction of dihydropyridine: 5.1 mg of Pd(P^{t}Bu₃)₂ (0.01 mmol, 5 mol %), 6.8 mg of the ligand L4 (0.011 mmol, 5.5 mol %), and 1 mL of the solvent toluene were added to a reaction flask and fully stirred; subsequently, 46.8 mg of (E)-3-hepten-2-yl carboxylate (0.3 mmol, 1.5 equiv), the well-stirred palladium catalyst solution, and 44.8 mg of 1,4-diazabicyclo[2.2.2]octane (0.3 mmol, 1.5 equiv) were added to a 1,4-dihydropyridine solution cooled to room temperature and stirred in a nitrogen atmosphere at 50° C. for 24 hours to obtain (S,E)-3-(3-hepten-2-yl)-5-phenyl-1-(4,4,5,5-tetramethyl-1,3,2-dioxaborolane)-1,4-dihydropyridin e, which was substituted with allyl in the meta-position, the reaction equation being as follows:

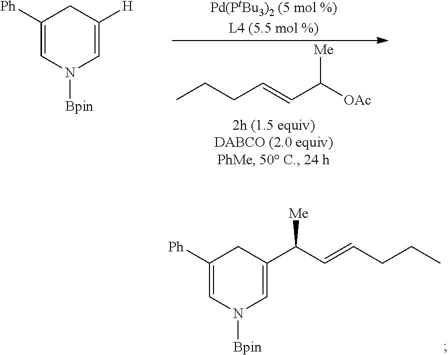

in which the structural formula of the ligand L4 was as follows:

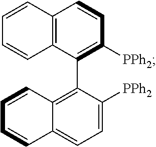

and
S3, oxidative aromatization to obtain a meta-allyl-substituted pyridine compound: the system was cooled to room temperature and stirred in air for 1 hour to generate (S,E)-3-(hept-3-en-2-yl)-5-phenylpyridine (4h), the reaction equation being as follows:

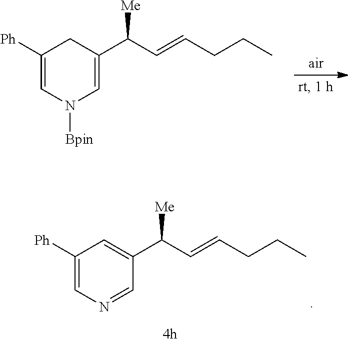

After the reaction was complete, the solvent was removed by rotary evaporation, and the residue was separated and purified by silica gel column chromatography to obtain the product (S,E)-3-(3-hepten-2-yl)-5-phenylpyridine as a colorless liquid, with a yield of 64%, an ee value of 92%, and an rr value of 5.4:1.

The product was characterized as follows:

¹H NMR (400 MHz, CDCl₃) δ 8.67 (d, J=1.9 Hz, 1H), 8.46 (d, J=1.6 Hz, 1H), 7.69 (t, J=1.9 Hz, 1H), 7.58 (d, J=7.3 Hz, 2H), 7.47 (t, J=7.5 Hz, 2H), 7.40 (t, J=7.3 Hz, 1H), 5.62 (dd, J=15.4, 6.5 Hz, 1H), 5.56-5.47 (m, 1H), 3.57-3.47 (m, 1H), 2.02 (dd, J=14.1, 7.0 Hz, 2H), 1.44-1.36 (m, 5H), 0.90 (t, J=7.3 Hz, 3H). ¹³C NMR (101 MHz, CDCl₃) δ 148.1, 146.1, 141.6, 138.3, 136.5, 134.0, 133.2, 130.5, 129.1, 128.1, 127.4, 40.0, 34.7, 22.7, 21.5, 13.8. HRMS (ESI) calcd. for C₁₈H₂₂N⁺ (M+H)⁺: 252.1747, found: 252.1749. HPLC retention times (IF, eluent: Hexanes/i-PrOH=98/2, 254 nm, flow rate: 0.3 mL/min, T=25° C.): t_{R}=51.1 min (minor), t_{R}=53.7 min (major).

### Example 19

A method for preparing (S,E)-3-(3-methyl-4-phenyl-3-buten-2-yl)-5-phenylpyridine, including the following steps:
S1, hydroboration reaction to prepare dihydropyridine: in a glove box filled with nitrogen, the catalyst B(2,4,6-F₃C₆H₂)₃ (LA1) (8.0 mg, 0.02 mmol, 10.0 mol %), 1 mL of toluene, 38.4 mg of pinacol borane (0.30 mmol, 1.5 equiv), and 31.0 mg of 3-phenylpyridine (1a) (0.2 mmol, 1.0 equiv) were added to an 8 mL small reaction flask, stirred, and reacted at 80° C. for 12 hours to obtain 3-phenyl-1-(4,4,5,5-tetramethyl-1,3,2-dioxaborolane)-1,4-dihydropyridine, the reaction equation being as follows:

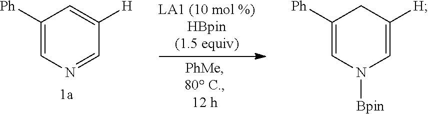

in which the structural formula of the catalyst LA1 was as follows:

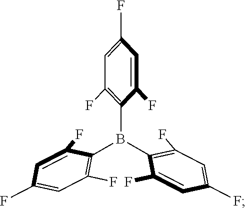

S2, palladium-catalyzed asymmetric allylation reaction of dihydropyridine: 5.1 mg of Pd(P^{t}Bu₃)₂ (0.01 mmol, 5 mol %), 6.8 mg of the ligand L4 (0.011 mmol, 5.5 mol %), and 1 mL of the solvent toluene were added to a reaction flask and fully stirred; subsequently, 61 mg of (E)-3-methyl-4-phenyl-3-buten-2-yl carboxylate (0.3 mmol, 1.5 equiv), the well-stirred palladium catalyst solution, and 44.8 mg of 1,4-diazabicyclo[2.2.2]octane (0.3 mmol, 1.5 equiv) were successively added to a 1,4-dihydropyridine solution cooled to room temperature and stirred in a nitrogen atmosphere at 60° C. for 24 hours to obtain (S,E)-3-(3-methyl-4-phenyl-3-buten-2-yl)-5-phenyl-1-(4,4,5,5-tetramethyl-1,3,2-dioxaborolane)-1,4-dihydropyridine, which was substituted with allyl in the meta-position, the reaction equation being as follows:

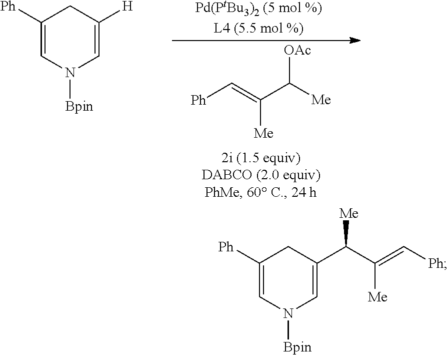

in which the structural formula of the ligand L4 was as follows:

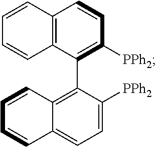

and
S3, oxidative aromatization to obtain a meta-allyl-substituted pyridine compound: the system was cooled to room temperature and stirred in air for 1 hour to generate (S,E)-3-(3-methyl-4-phenylbut-3-en-2-yl)-5-phenylpyridine (4i), the reaction equation being as follows:

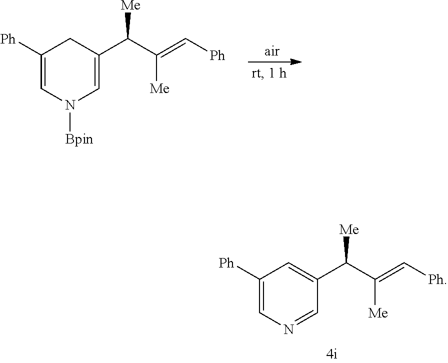

After the reaction was complete, the solvent was removed by rotary evaporation, and the residue was separated and purified by silica gel column chromatography to obtain the product (S,E)-3-(3-methyl-4-phenyl-3-buten-2-yl)-5-phenylpyridine as a colorless liquid, with a yield of 57% and an ee value of 91%.

The product was characterized as follows:
[α]²⁹D=+42.8 (c=1.05, CHCl₃). ¹H NMR (400 MHz, CDCl₃) δ 8.70 (d, J=2.1 Hz, 1H), 8.54 (d, J=2.1 Hz, 1H), 7.76 (t, J=2.1 Hz, 1H), 7.61-7.56 (m, 2H), 7.51-7.44 (m, 2H), 7.43-7.37 (m, 1H), 7.36-7.30 (m, 2H), 7.29-7.26 (m, 2H), 7.24-7.18 (m, 1H), 6.54 (s, 1H), 3.73-3.63 (m, 1H), 1.76 (d, J=1.1 Hz, 3H), 1.56 (d, J=7.1 Hz, 3H). ¹³C NMR (101 MHz, CDCl₃) δ 148.4, 146.4, 141.1, 140.2, 138.1, 136.5, 133.4, 129.2, 129.1, 128.2, 128.2, 127.4, 126.4, 125.8, 46.2, 19.5, 16.7. HRMS (ESI) calcd. for C₂₂H₂₂N⁺ (M+H)⁺: 300.1747, found: 300.1741. HPLC retention times (IA-3, eluent: Hexanes/i-PrOH=97/3, 254 nm, flow rate: 0.5 mL/min, T=25° C.): t_{R}=26.8 min (major), t_{R}=29.0 min (minor)

### Examples 20-25

The following examples were all methods for preparing (R)-3-phenyl-5-(1-p-tolylallyl)pyridine. The steps and reaction conditions thereof were approximately the same as those in Example 1, except for the following differences:

| | Boron | Iridium | | Yield of | ee value |
|---|---|---|---|---|---|
| Example | catalyst | catalyst | Ligand | 3a (%) | of 3a (%) |
| 20 | LA2 | [Ir(COD)Cl]₂ | L1 | 77 | 98 |
| 21 | LA3 | [Ir(COD)Cl]₂ | L1 | 76 | 98 |
| 22 | LA1 | [Ir(COD)OMe]₂ | L1 | 77 | 98 |
| 23 | LA1 | Ir(COD)BF₄ | L1 | 69 | 99 |
| 24 | LA1 | [Ir(COD)Cl]₂ | L2 | 64 | 96 |
| 25 | LA1 | [Ir(COD)Cl]₂ | L3 | 57 | 93 |

wherein the amount of the catalyst LA2 was 6.5 mg (0.01 mmol, 5 mol %), the amount of LA3 was 2.4 mg (0.01 mmol, 5 mol %), and the structural formulas of the two catalysts were respectively as follows:

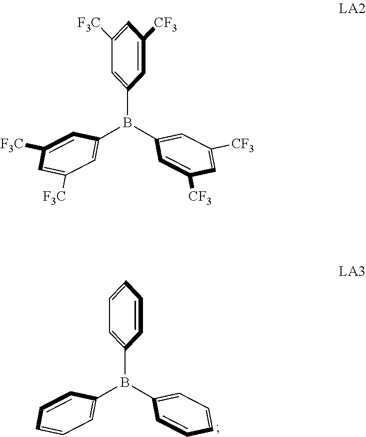

wherein the amount of the ligand L1 was 6.3 mg (0.011 mmol, 5.5 mol %), the amount of the ligand L2 was 5.5 mg (0.011 mmol, 5.5 mol %), the amount of the ligand L3 was 5.1 mg (0.011 mmol, 5.5 mol %), and the structural formulas of the three ligands were respectively as follows:

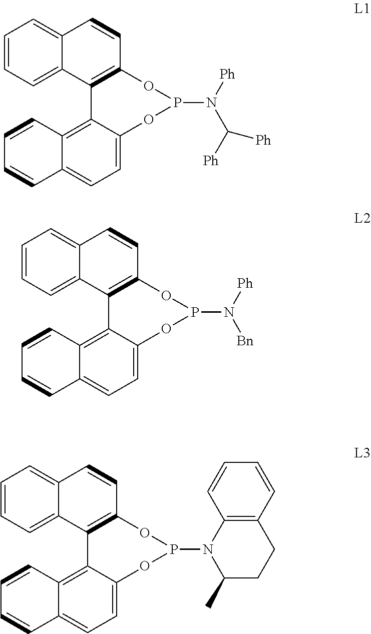

### Examples 21-32

The following examples were all methods for preparing (R)-3-phenyl-5-(1-p-tolyl allyl)pyri dine. The steps and reaction conditions thereof were approximately the same as those in Example 1, except for the following differences:

| | | | | | ee |
|---|---|---|---|---|---|
| | | | | Yield | value |
| | Allyl | | | of 3a | of 3a |
| Example | ester | Solvent | Alkali | (%) | (%) |
| 21 | 2a-OAc | Tetrahydrofuran | 1,4-Diazabicyclo[2.2.2]octane | 70 | 97 |
| 22 | 2a-OBoc | Tetrahydrofuran | 1,4-Diazabicyclo[2.2.2]octane | 13 | 99 |
| 23 | 2a-OCO₂Me | Tetrahydrofuran | 1,4-Diazabicyclo[2.2.2]octane | 9 | 90 |
| 24 | 2a-OBz | Tetrahydrofuran | 1,4-Diazabicyclo[2.2.2]octane | 73 | 94 |
| 25 | 2a | Dichloromethane | 1,4-Diazabicyclo[2.2.2]octane | 68 | 98 |
| 26 | 2a | Toluene | 1,4-Diazabicyclo[2.2.2]octane | 65 | 99 |
| 28 | 2a | 1,4-Dioxane | 1,4-Diazabicyclo[2.2.2]octane | 49 | 98 |
| 29 | 2a | Tetrahydrofuran | Triethylamine | 68 | 96 |
| 30 | 2a | Tetrahydrofuran | 1,8-Diazabicycloundec-7-ene | 54 | 97 |
| 31 | 2a | Tetrahydrofuran | N-ethylpiperidine | 50 | 96 |
| 32 | 2a | Tetrahydrofuran | Cesium carbonate | 45 | 97 |

wherein the amount of the allyl ester 2a was 130.9 mg (0.6 mmol, 3.0 equiv), the amount of 2a-OAc was 105.6 mg (0.6 mmol, 3.0 equiv), the amount of 2a-OBz was 142.8 mg (0.6 mmol, 3.0 equiv), the amount of 2a-OCO₂Me was 115.2 mg (0.6 mmol, 3.0 equiv), the amount of 2a-OBoc was 140.4 mg (0.6 mmol, 3.0 equiv), and the structural formulas of the five allyl esters were respectively as follows:

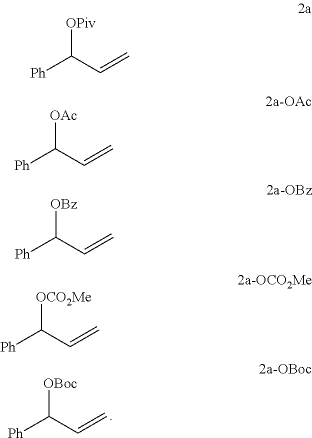

What is claimed is:

1. A method for preparing a meta-(chiral allyl)-substituted pyridine compound, comprising the following steps:
S1, preparation of dihydropyridine:
in a glove box filled with nitrogen, adding pyridine, a catalyst, a solvent, and pinacol borane to a reaction flask and fully reacting a mixture under stirring to obtain 1,4-dihydropyridine or 1,2-dihydropyridine, a reaction equation being as follows: 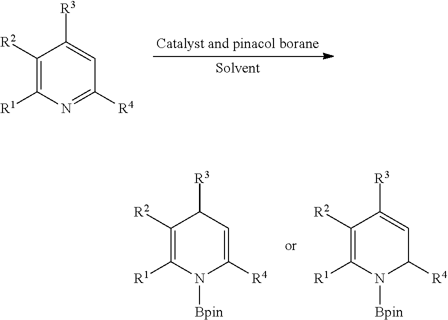
wherein:
the catalyst is triarylboron, a molar ratio of the triarylboron to the pyridine is (2.5-10):100, and a structural formula of the triarylboron is B(R⁵)₃ in which R⁵ is phenyl, 3,5-bis(trifluoromethyl)-substituted phenyl, or 2,4,6-trifluoro-substituted phenyl;
an equivalent ratio of the pinacol borane to the pyridine is (1-2):1;
R¹ is hydrogen, alkyl, aryl, alkenyl, substituted aryl, or heteroaryl;
R² is hydrogen, alkyl, halogen, amino, ether group, ester group, aryl, alkenyl, alkynyl, substituted aryl, or heteroaryl;
R³ is hydrogen, alkyl, aryl, or substituted aryl; and
R⁴ is hydrogen, alkyl, aryl, alkenyl, substituted aryl, or heteroaryl;
S2, metal-catalyzed asymmetric allylation reaction of thiopyridine:
in the glove box filled with the nitrogen, adding a metal catalyst, a chiral ligand, and a solvent to a reaction flask, fully stirring a mixture, then adding an alkali, stirring a mixture, then adding an allyl reagent, finally adding the 1,4-dihydropyridine or the 1,2-dihydropyridine prepared in the step S1, which has been cooled to room temperature, and stirring a mixture until the reaction is complete, to obtain a dihydropyridine substituted with chiral allyl in a meta-position, a reaction equation being as follows: 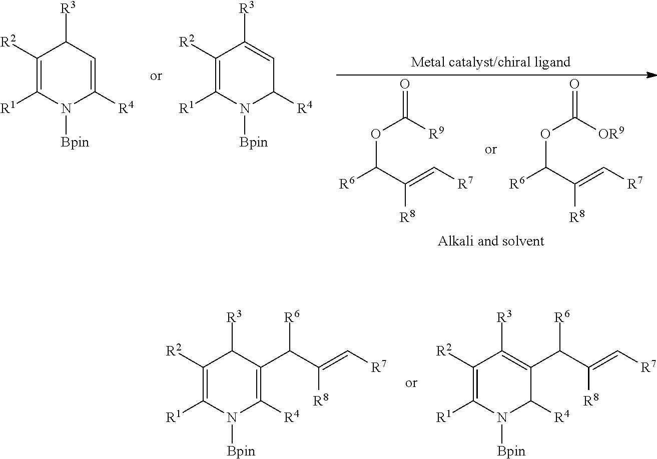
wherein
the metal catalyst is selected from any one of [Ir(COD)Cl]₂, [Ir(COD)OMe]₂, Ir(COD)BF₄, and Pd(P^{t}Bu₃)₂, and a molar ratio of the metal catalyst to the pyridine is (1-10):100;
a structural formula of the chiral ligand is as follows: 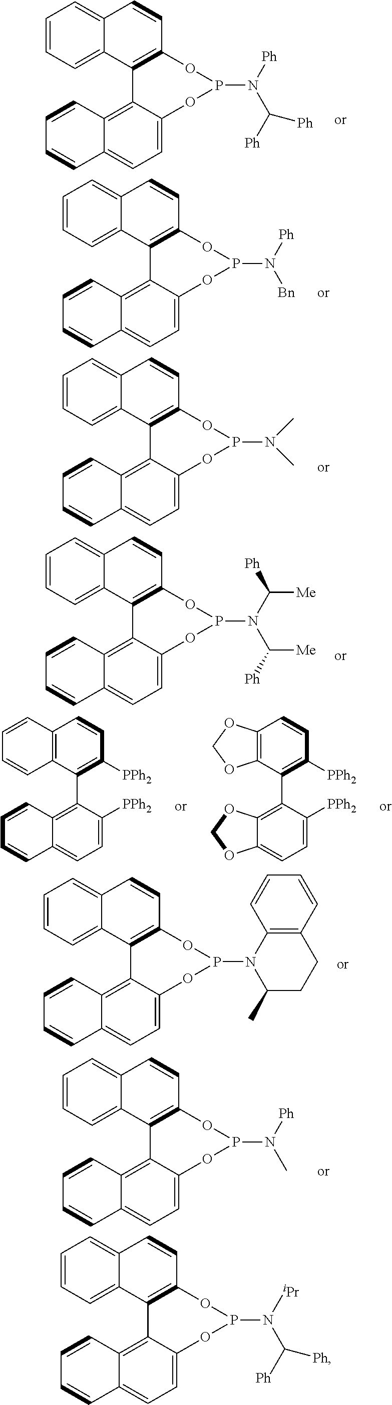
a molar ratio of the chiral ligand to the pyridine is (1-20):100;
the alkali is triethylamine, diisopropylethylamine, 1,5,7-triazabicyclo[4.4.0]dec-5-ene, 1,5-diazabicyclo[5.4.0]-5-undecene, N-ethylpiperidine, 1,4-diazabicyclo[2.2.2]octane, sodium carbonate, or cesium carbonate, and an equivalent ratio of the alkali to the pyridine is (1-200):100;
the allyl reagent is allyl carboxylate or allyl carbonate, and an equivalent ratio of the allyl reagent to the pyridine is (1-5):1;
R⁶ is alkyl, aryl, alkenyl, substituted aryl, or heteroaryl;
R⁷ is hydrogen, alkyl, alkenyl, alkynyl, aryl, substituted aryl, or heteroaryl;
R⁸ is hydrogen or alkyl;
R⁹ is aryl or alkyl; and
a reaction time of the reaction is 16-24 hours; and
S3, oxidative aromatization to obtain the meta-(chiral allyl)-substituted pyridine compound:
placing the reaction flask in the step S2 in air, stirring the mixture until an oxidative aromatization reaction is complete, performing distillation under reduced pressure to remove the solvent and then separation by column chromatography to obtain the meta-(chiral allyl)-substituted pyridine compound, a reaction equation being as follows: 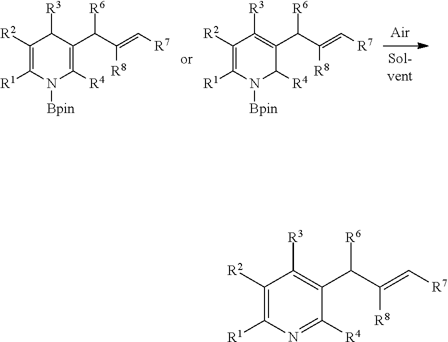
wherein a reaction temperature is room temperature and a reaction time is 1 minute to 24 hours; and
solvents in the steps S1 and S2 are the same and are both selected from at least one of trichloromethane, dichloromethane, 1,2-dichloroethane, ethyl acetate, ethylene glycol dimethyl ether, methyl tert-butyl ether, 1,4-dioxane, methyl cyclopentyl ether, methyl tetrahydrofuran, tetrahydrofuran, toluene, trifluorotoluene, o-xylene, m-xylene, p-xylene, deuterated chloroform, or deuterated tetrahydrofuran.

2. The method according to claim 1, wherein in the step S2, the allyl reagent is:

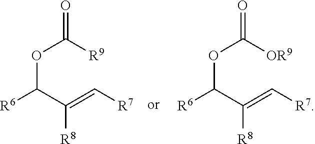

3. The method according to claim 2, wherein the equivalent ratio of the allyl reagent 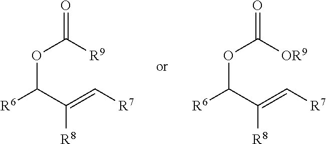
or to the pyridine is 1.5:1-3:1.

4. The method according to claim 1, wherein in the step S1, a reaction temperature is from room temperature to 120° C., and a reaction time is 1-12 hours; and in the step S2, a reaction temperature is −10° C. to 60° C.

5. The method according to claim 3, wherein the reaction temperature in the step S1 is 40° C.-80° C., and the reaction temperature in the step S2 is 30° C.-60° C.

6. The method according to claim 1, wherein in the step S1, when the R¹ is the heteroaryl, the heteroaryl is furyl or thienyl; when the R² is the heteroaryl, the heteroaryl is furyl, thienyl, benzothiophenyl, benzofuryl, or pyridinyl; and when the R⁴ is the heteroaryl, the heteroaryl is furyl or thienyl.

7. The method according to claim 1, wherein in the step S1, the equivalent ratio of the pinacol borane to the pyridine is 1.5:1.

8. The method according to claim 1, wherein in the step S2, when the R⁶ is the heteroaryl, the heteroaryl is furyl, thienyl, pyridinyl, or indolyl; and when the R⁷ is the alkyl, the alkyl is propyl, butyl, pentyl, or cyclohexyl.

9. The method according to claim 1, wherein in the step S2, when the R⁷ is the heteroaryl, the heteroaryl is furyl, thienyl, or pyridinyl.

10. The method according to claim 1, wherein in the step S2, the alkali is preferably the 1,4-diazabicyclo[2.2.2]octane, and the preferred equivalent ratio of the alkali to the pyridine is 1:1.
